SEQUENCE LISTING (18 sequences)

SEQ ID NO: 1 - DNA, 21 bp, synthetic construct
  misc_feature 1..21: note = synthetic sequence
  source 1..21: mol_type = other DNA
catccagagcgtccctggctt

SEQ ID NO: 2 - DNA, 20 bp, synthetic construct
  misc_feature 1..20: note = synthetic sequence
  source 1..20: mol_type = other DNA
ggccgaagaaacactgagaa

SEQ ID NO: 3 - DNA, 43 bp, synthetic construct
  misc_feature 1..43: note = synthetic sequence
  source 1..43: mol_type = other DNA
ttgggacaccggaaagatgtaagatgagtctagactcgtggtg

SEQ ID NO: 4 - DNA, 48 bp, synthetic construct
  misc_feature 1..48: note = synthetic sequence
  source 1..48: mol_type = other DNA
agaacatccggatctatatggtctgcccctagaaaattgagagaagtc

SEQ ID NO: 5 - DNA, 22 bp, synthetic construct
  misc_feature 1..22: note = synthetic sequence
  source 1..22: mol_type = other DNA
ggacggagatgaaggtctacta

SEQ ID NO: 6 - DNA, 21 bp, synthetic construct
  misc_feature 1..21: note = synthetic sequence
  source 1..21: mol_type = other DNA
gggaggtctccaccctattta

SEQ ID NO: 7 - DNA, 131 bp, synthetic construct
  misc_feature 1..131: note = synthetic sequence
  source 1..131: mol_type = other DNA
gaccgtcaattcaccgaggacggagatgaaggtctactactctccgtactgccgaggggccagataacggttgaattcgtgagagctatcccctaaatagggtggagacctccctcggtgaattgacggtc

SEQ ID NO: 8 - DNA, 15 bp, synthetic construct
  misc_feature 1..15: note = synthetic sequence
  source 1..15: mol_type = other DNA
aggggctgggagggc

SEQ ID NO: 9 - DNA, 15 bp, synthetic construct
  misc_feature 1..15: note = synthetic sequence
  source 1..15: mol_type = other DNA
cctcccgggtccccg

SEQ ID NO: 10 - DNA, 11 bp, synthetic construct
  misc_feature 1..11: note = synthetic sequence
  misc_feature 2: note = bridged nucleic acid
  misc_feature 4: note = bridged nucleic acid
  misc_feature 5: note = bridged nucleic acid
  misc_feature 6: note = bridged nucleic acid
  misc_feature 7: note = bridged nucleic acid
  misc_feature 8: note = bridged nucleic acid
  misc_feature 10: note = bridged nucleic acid
  misc_feature 11: note = phosphorylation modification
  source 1..11: mol_type = other DNA
gccccctccgg

SEQ ID NO: 11 - DNA, 12 bp, synthetic construct
  misc_feature 1..12: note = synthetic sequence
  misc_feature 1: note = fluorescent tag 6FAM
  misc_feature 3: note = locked nucleic acid
  misc_feature 5: note = locked nucleic acid
  misc_feature 8: note = locked nucleic acid
  misc_feature 11: note = locked nucleic acid
  source 1..12: mol_type = other DNA
cagccccttccg

SEQ ID NO: 12 - DNA, 18 bp, synthetic construct
  misc_feature 1..18: note = synthetic sequence
  source 1..18: mol_type = other DNA
aaatacgggtattttcgc

SEQ ID NO: 13 - DNA, 16 bp, synthetic construct
  misc_feature 1..16: note = synthetic sequence
  source 1..16: mol_type = other DNA
aacgaccacaacgacg

SEQ ID NO: 14 - DNA, 14 bp, synthetic construct
  misc_feature 1..14: note = synthetic sequence
  misc_feature 1: note = fluorescent tage 6FAM
  misc_feature 2: note = locked nucleic acid
  misc_feature 4: note = locked nucleic acid
  misc_feature 8: note = locked nucleic acid
  misc_feature 11: note = locked nucleic acid
  misc_feature 14: note = locked nucleic acid
  source 1..14: mol_type = other DNA
tgttttgcggtcgt

SEQ ID NO: 15 - DNA, 46 bp, synthetic construct
  misc_feature 1..46: note = synthetic sequence
  source 1..46: mol_type = other DNA
gctcttcgtggtgtggtgtccatataaacttgtggtagttggacct

SEQ ID NO: 16 - DNA, 41 bp, synthetic construct
  misc_feature 1..41: note = synthetic sequence
  source 1..41: mol_type = other DNA
gctcttcgtggtgtggtgtcccgtccacaaaatgattctga

SEQ ID NO: 17 - DNA, 30 bp, synthetic construct
  misc_feature 1..30: note = synthetic sequence
  source 1..30: mol_type = other DNA
actgaatataaacttgtggtagttggacct

SEQ ID NO: 18 - DNA, 25 bp, synthetic construct
  misc_feature 1..25: note = synthetic sequence
  source 1..25: mol_type = other DNA
gtccacaaaatgatcctggattagc

(19) United States
(12) Patent Application Publication
(10) Pub. No.: US 2023/0183672 A1
(43) Pub. Date: Jun. 15, 2023

(54) METHODS FOR ISOLATING CIRCULATING NUCLEIC ACIDS FROM URINE SAMPLES

(71) Applicant: JBS Science Inc., Doylestown, PA (US)

(72) Inventors: Zhili WANG, Doylestown, PA (US); Wei SONG, Doylestown, PA (US); Selena LIN, Doylestown, PA (US)

(73) Assignee: JBS Science Inc., Doylestown, PA (US)

(21) Appl. No.: 17/813,570

(22) Filed: Jul. 19, 2022

Related U.S. Application Data
(60) Provisional application No. 63/223,542, filed on Jul. 19, 2021.

(51) Int. Cl. C12N 15/10; C12Q 1/6886; C12Q 1/6879; C12Q 1/70; B01D 21/26

(52) U.S. Cl. CPC C12N 15/1003; C12Q 1/6886; C12Q 1/6879; C12Q 1/706; B01D 21/262; C12Q 2600/156; C12Q 2600/154

(57) ABSTRACT

Provided herein includes a method for characterizing a target cell-free nucleic acid (cfNA) molecule present in a biological sample such as a urine sample. It comprises isolating total cfNAs from the biological sample without prior preprocessing such as centrifugation to remove cell debris, and characterizing the target cfNA molecule based on the isolated total cfNAs. When the target cfNA is a low molecular weight (LMW) molecule, the method additionally comprises a fractionation step to obtain LMW nucleic acids from the total cfNAs before characterization. The method can detect significantly more copies of the target cfNA molecule compared with existing methods which typically discard the cell debris from the biological sample. Another method is also provided, which substantially recovers cfNAs from the usually discarded cell debris, thus also capable of detecting significantly more copies of the target cfNA molecule.

### CROSS-REFERENCE TO RELATED APPLICATION

The present application claims the benefit of U.S. Provisional Application No. 63/223,542 filed on Jul. 19, 2021, the disclosure of which is hereby incorporated by reference in its entirety.

### REFERENCE TO SEQUENCE LISTING SUBMITTED ELECTRONICALLY

The content of the electronically submitted sequence listing, with a file name SeqListing.xml, size 30,375 bytes, and date of creation Jul. 19, 2022, filed herewith, is incorporated herein by reference in its entirety.

### BACKGROUND

The presence of circulatory nucleic acids (NAs), such as circulating DNAs (e.g. cell-free DNAs, i.e. cfDNAs) or circulating RNAs (e.g. cell free RNAs, i.e. cfRNAs) in urine is well established. For example, it has been shown that in a pregnant woman, extracellular fetal DNA is present in the maternal circulation and can be detected not only in the maternal blood, but also in the maternal urine sample, although in much shorter in length and much less concentration (Chan et al. 2003; Tsui et al. 2012; Botezatu et al. 2000; Al-Yatama et al. 2001; Majer et al. 2007; Li et al. 2003; Illanes et al. 2006; Koide et al. 2005; Shekhtman et al. 2009). These findings clearly limit the potential clinical utilities of urine DNA as suggested by previous studies. Interestingly, most of the cited studies isolated urine cfDNA from the maternal urine sample were subjected to centrifugation to remove cell pellets as a pre-processing step before DNA isolation which is similar to current existing approaches for recovering cfNA from the urine samples. Studies have shown that the circulatory fetal genetic material can be used for the reliable determination, e.g. by PCR (polymerase chain reaction) technology, of fetal genetic loci which are completely absent from the maternal genome with examples of such fetal genetic loci that have been successfully identified in in maternal urine is fetal Y chromosome-specific sequences (Tsui et al. 2012; Al-Yatama et al. 2001; Lin et al. Diagnostics 2021. 11(4)). Studies by us also shown detection of liver derived hepatitis B virus (HBV) DNA, hepatocellular carcinoma DNA biomarkers, and colorectal cancer (CRC) DNA biomarkers in urine samples of patients with hepatitis B virus infections (Lin et al. Hepatology communication 2022; Jain et al. 2018), HCC (Lin et al. Diagnostics 2021. 11(8); Hann et al. 2017; Zhang et al. 2018; Kim et al. 2022), and CRC (Botezatu et al. 2000; Su et al. 2004; Su et al. 2005; Song et al. 2012), respectively.

Currently, almost all existing approaches including commercially available kits for recovering cfNAs from the urine sample rely on the pre-processing using centrifugation, e.g. at 1,000 rpm for 10 min or a higher speed for a shorter time, prior to DNA isolation, so as to remove any cell debris in order to achieve a better DNA isolation efficiency. However these existing cfDNA recovery approaches suffer from poor recovery rates (Chan et al. 2003; Tsui et al. 2012) even though the isolation methods are suitable for blood cfNA isolations.

### SUMMARY OF THE INVENTION

In order to address the aforementioned issues associated with existing cfNA detection approaches, the present disclosure provides methods for characterizing a target cell-free nucleic acid (cfNA) molecule present in a biological sample, such as the quite challenging urine sample.

One such method provided herein substantially comprises the following two steps:

(1) isolating total cell-free nucleic acids from the biological sample without prior preprocessing the biological sample that removes cell debris therefrom (e.g. by means of centrifugation); and

(2) characterizing the target cell-free nucleic acid molecule from the isolated total cell-free nucleic acids obtained from step (1).

Herein the method is capable of detecting at least 2 fold, and preferably at leat 6 fold, copies of the target cell-free nuclei acid molecule in the biological sample compared with when the cell debris is removed in the prior preprocessing in the isolating step (1).

When the target cfNA molecule is a low molecular weight (LMW) DNA, i.e. having a length shorter than approximately 1 kb, in order to increase the detection sensitivity, the method further comprises a step of obtaining low-molecular weight DNAs from the isolated total cell-free DNAs after the isolating total cell-free DNAs from the urine sample and before the characterizing the target cell-free DNA molecule from the isolated total cell-free DNAs. Correspondingly, the step of characterizing the target cell-free DNA molecule from the isolated total cell-free DNAs comprises: characterizing the target cell-free DNA molecule from the low-molecular weight DNAs.

Another such method provided herein comprises the following three major steps:

(a) preprocessing the biological sample, which specifically comprises: centrifugating the biological sample to thereby obtain a supernatant fraction and a pellet fraction; washing the pellet fraction using a wash solution to thereby obtain a wash-off fraction; and combining the supernatant fraction and the wash-off fraction to thereby obtain a combined fraction;

(b) isolating total cell-free nucleic acids from the combined fraction obtained from step (a); and

(c) characterizing the target cell-free nucleic acid molecule from the isolated total cell-free nucleic acids obtained from step (b).

Herein, the method is capable of detecting at least 1.25 fold, or preferably at least 2 fold copies of the target cell-free nucleic acid molecule from the biological sample compared with when the total cell-free nucleic acids are isolated only from the supernatant fraction in the preprocessed biological sample.

More details will be provided below.

### BRIEF DESCRIPTION OF THE DRAWINGS

FIG. 1 shows the association of transrenal Y-Chr DNA with cell pellet after centrifugation. Briefly, 10 mL each from different collections of urine from two donors (donor 1 and 2) of pregnant mother with male fetus were spiked with 1×10⁵ copies/mL of synthetic double stranded DNA, Artificial Spike-In (SPKN) each, centrifuge at 1,500 rpm for 10 min at 4C to fractionate supernatant and pellet. DNA was isolated, eluted, and quantitated by qPCR assays for spiked control SPKN and Y-Chr (Lin et al. Diagnostics 2021. 11(4)). The percent of recovery from SPKN input and percent of Y-Chr total recovery were calculated and plotted.

FIG. 2 shows the association of transrenal HBV DNA with cell pellet after centrifugation. Briefly, 10 mL each from different collections of urine from four donors (donor 1-3) of patients with chronic HBV infection were spiked with 1×10⁵ copies/mL of synthetic double stranded DNA, SPKN each, centrifuge at 1,500 rpm for 10 min at 4C to fractionate supernatant and pellet. DNA was isolated, eluted, and quantitated by qPCR assays for spiked control SPKN and HBV pol/S assay, as detailed in Table 1. The percent of recovery from SPKN input and percent of HBV DNA total recovery were calculated and plotted.

FIG. 3 demonstrates enhanced detection sensitivity of CRC-associated K-ras mutation by preferential isolation of low-MW urine DNA from total urine DNA using gel electrophoresis. Briefly, total and low-MW urine DNA were prepared as described in text and subjected to the RE-PCR assay for mutated K-ras DNA. The photos shown represent the difference in the outcome of RE-PCR between total and low-MW urine DNA from 6 different individuals with diagnosed colorectal cancer. Fold increase of detection sensitivity in Low-MW DNA was indicated.

FIG. 4 shows an outline of the experimental procedures for collecting urine cfDNAs in the urine samples from patients with hepatocellular carcinoma (HCC) so as to examine the detection sensitivities in detecting two HCC-associated DNA markers: aberrant methylation of RASSF1A (mRASSF1A) gene and hTERT-124 hot spot mutation (mTERT) using different methods.

### DETAILED DESCRIPTION

In a first aspect, the present disclosure provides a first method for characterizing a target cell-free nucleic acid molecule present in a biological sample. The first method substantially comprises the following two steps:

(1) isolating total cell-free nucleic acids from the biological sample without prior preprocessing the biological sample that removes cell debris therefrom; and

(2) characterizing the target cell-free nucleic acid molecule from the isolated total cell-free nucleic acids obtained from step (1).

Herein the method is capable of detecting at least 2 fold copies of the target cell-free nuclei acid molecule in the biological sample compared with when the cell debris is removed in the prior preprocessing in the isolating step (1).

As used herein and throughout the whole disclosure, the term “biological sample” is referred to as a sample, including one or a combination of a urine sample, a serum sample, a plasma sample, a saliva sample, a sweat sample, or a lymph liquid sample, that are obtained from one or more biological subjects. These biological samples contain nucleic acids but generally contain no cells. Such biological sample could be from a single source (e.g. urine sample) or from a combination of multiple sources, such as a combination of a urine sample and a plasma sample. Such biological sample could be obtained from a single subject (e.g. a cancer patient or a pregnant female), or could be a pooled sample from more than one subject. Such biological sample can be freshly obtained, or can be thawed from a frozen sample, or can be a processed biological sample as long as such processing does not remove the cell debris therefrom.

As used herein, the term “nucleic acid” is referred to as DNA and/or RNA molecule(s), and the term “cell-free nucleic acid”, “circulating nucleic acid” or alike, is referred to as one or more nucleic acid molecules that exist or are present in one particular biolgical sample (e.g. urine sample or plasma sample) as defined above. For example, a cell-free nucleic acid present in the urine sample may comprise a transrenal DNA (i.e. DNA deriving originally in the circulation may pass the renal filtration to thereby become a transrenal DNA), or may comprise an apoptosis-derived nucleic acid molecule (i.e. derived from apoptotic cells from the urinary tract of a subject wherefrom the urine sample is obtained). In another example, a cell-free nucleic acid in a plasam sample may derive from cell-free nucleic acids that coexisent with other blood cells in the circulation. Herein, the target cell-free nuclei acid molecule can be a DNA (e.g. cell-free DNA or cfDNA, etc.) molecule or an RNA molecule (e.g. microRNA or miRNA, cell-free RNA or cfRNA, etc.).

In the above isolating step (1), the phrase “prior preprocessing the biological sample that removes cell debris therefrom” means a preprocessing step on or over the biological sample that is performed before the isolating step (1), and by means of this preprocessing step, the cell debris contained in the biological sample is removed therefrom. One common example of such preprocessing step may typically include centrifugating the biological sample (e.g. at 1,500 RPM at 4° C. for 10 minutes), which is traditionally applied in almost all existing target cell-free nucleic acid marker characterization method with a purpose to provide a cleaner biological sample for the convenience and effectiveness for nucleic acid isolation and target marker detection. Yet it is noted that such cell-debris-removing preprocessing step is not limited to centrifugation, and can include other means or approaches as well.

In the above characterizing step (2), “characterizing”, “characterize”, “characterization” or alike, is construed to include either one or both of qualification (i.e. detecting or monitoring if a target nuclei acid molecule of interest is present or absent in the biological sample) and quantification (i.e. determining the level, such as copy number, weight, ratio, concentration, etc. of the target cell-free nuclei acid molecule of interest is present or absent in the biological sample).

Herein the method is capable of detecting at least 2 fold, and more preferably at least 6 fold, copies of the target cell-free nuclei acid molecule in the biological sample compared with when the cell debris is removed in the prior preprocessing in the isolating step (1).

As demonstrated in the Examples provided below, where the total cell-free nucleic acids are extracted directly from the biological sample (i.e. urine sample), the capability of detecting the target cell-free DNA markers could be greatly elevated (up to approximately 30 fold) compared with their corresponding control experiments where the biological sample undergoes traditional centrifucation preprocessing and only the post-centrifugation supernatant fraction is used for DNA isolation and target marker detection.

For example, in Example 3 as shown below, where the urine samples from pregnant mothers were tested for fetal Y-Chr DNA detection capabilities, it is demonstrated that as dramatic as 6.2 fold and 11.7 fold copies of such markers can be detected in the urine sample by the method that involves no centrifugation (i.e. “N.C.” group) compared with the traditional method (i.e. the “supernatant” group in the “Centrifugation” group) (see Table 2).

In another example shown in Example 4 below, where the urine samples from hepatocellular carcinoma (HCC) patients were tested for HCC markers (i.e. methylated RASSF1A (mRASSF1A) and/or mutated hTERT (mTERT)) detection capabilities, it is independently demonstrated that as dramatic as 7.1 fold and 29.2 fold copies of the mRASSF1A marker can be detected in the urine sample by the method that involves no centrifugation (i.e. “N.C.” group) compared with the traditional method (i.e. the “supernatant” group in the “Centrifugation” group) (see Table 3), and regarding the mTERT marker, at least 6 fold copies of the marker can be found using the first method as provided herein compared with the traditional method.

According to some embodiments of the method provided in this first aspect, the biological sample is a urine sample, and the target cell-free nucleic acid molecule comprises at least one of a trans-renal nuclei acid molecule or an apoptosis-derived nucleic acid molecule.

Herein optionally, the target cell-free nucleic acid molecule is a target cell-free DNA molecule, and correspondingly, the first method comprises:

isolating total cell-free DNAs from the urine sample; and

characterizing the target cell-free DNA molecule from the isolated total cell-free DNAs.

According to some embodiments, the target cell-free DNA molecule is a low molecular weight (LMW) DNA, i.e. having a length shorter than approximately 1 kb, in order to increase the detection sensitivity, the method further comprises a step of obtaining low-molecular weight DNAs from the isolated total cell-free DNAs after the isolating total cell-free DNAs from the urine sample and before the characterizing the target cell-free DNA molecule from the isolated total cell-free DNAs. Correspondingly, the step of characterizing the target cell-free DNA molecule from the isolated total cell-free DNAs comprises: characterizing the target cell-free DNA molecule from the low-molecular weight DNAs.

Herein, the step of obtaining low-molecular weight DNAs from the isolated total DNAs can be by means of a size differentiation approach, which is optionally selected from a carboxylated magnetic beads-based approach, a chromatography approach based on an agarose gel, or a chromatography approach based on a polyacrylamide gel.

Herein, the step of characterizing the target cell-free DNA molecule from the low-molecular weight DNAs can optionally be realized by means of at least one of a polymerase chain reaction (PCR) assay, a sequencing assay, or a hybridization assay. Examples for the PCR assay may include a regular PCR assay, a real-time PCR assay, a quantitative PCR assay, etc. Examples for the sequencing assay may include Sanger sequencing, or next-generation sequencing. Examples for the hybridization assay may include a Southern blot assay, a microArray assay. These above assays may further include the use of primers, probes, etc. having sequences designed specficially to the target cell-free DNAs.

According to some embodiments as demonstrated below in Examples 2 and 4, the target cell-free DNA molecule is a cancer-associated DNA marker, selected from a mutant K-ras, methylated RASSF1A (mRASSF1A), or mutated TERT (mTERT).

According to some embodiments as demonstrated below in Examples 1 and 3, the urine sample is obtained from a pregnant female, and the target cell-free DNA molecule is a fetal DNA marker that is associated with sex, an autosomal trait, or a genetic disorder. Herein, the fetal DNA marker may optionally comprise a Y-Chromosome (Y-Chr) marker. Herein, the autosomal trait may include fetal RhD status (e.g. in the RhD-negative women), the genetic disorder may include male-sex-linked diseases, adrenal hyperplasia, myotonic dystrophy, achondroplasia, fetal aneuploidy, etc.

According to some embodiments as demonstrated below in Example 1, the target cell-free DNA molecule comprises an HBV DNA marker. Optionally, the target cell-free DNA molecule may be a DNA marker for other viruses (e.g. HIV, HCV, Covid19, SARS, etc.), or may be a DNA marker for other micro-organisms such as bacteria, or fungi.

Optionally in the method provided herein, the target cell-free nucleic acid molecule is a target cell-free RNA molecule, and correspondingly, the first method comprises:

isolating total cell-free RNAs from the urine sample; and

characterizing the target cell-free RNA molecule from the isolated total cell-free RNAs.

According to some embodiments, the target cell-free RNA molecule comprises a microRNA.

Herein, the step of characterizing the target cell-free DNA molecule from the low-molecular weight DNAs can optionally be realized by means of at least one of a reverse transcription polymerase chain reaction (RT-PCR) assay, a RNA sequencing assay, or a hybridization assay (e.g. Northern blot or microArray assay).

In any of the embodiments of the method as described above, the step of isolating total cell-free nucleic acids from the biological sample can optionally be performed with use of carrier RNAs, such as tRNAs.

In a second aspect, the present disclosure further provides a second method for characterizing a target cell-free nucleic acid molecule in a biological sample. Compared with the first method as described above in the first aspect, the second method still includes a centrifugation preprocessing step that removes the cell debris. More specifically, the method comprises the following three major steps:

(a) preprocessing the biological sample, which specifically comprises: centrifugating the biological sample to thereby obtain a supernatant fraction and a pellet fraction; washing the pellet fraction using a wash solution to thereby obtain a wash-off fraction; and combining the supernatant fraction and the wash-off fraction to thereby obtain a combined fraction;

(b) isolating total cell-free nucleic acids from the combined fraction obtained from step (a); and

(c) characterizing the target cell-free nucleic acid molecule from the isolated total cell-free nucleic acids obtained from step (b).

Herein, the method is capable of detecting at least 1.25 fold, or preferably at least 2 fold copies of the target cell-free nucleic acid molecule from the biological sample compared with when the total cell-free nucleic acids are isolated only from the supernatant fraction in the preprocessed biological sample.

As demonstrated in the Examples provided below, where the urine samples that have been centrifugation-preprocessed before the supernatant fraction and the pellet fraction undergo separate DNA marker detection, the capability of detecting the target cell-free DNA markers could be elevated (up to approximately 20 fold) if both fractions are combined for detection (i.e. “combination approach”) compared with control experiments where only the supernatant fraction is tested (i.e. “traditional approach”).

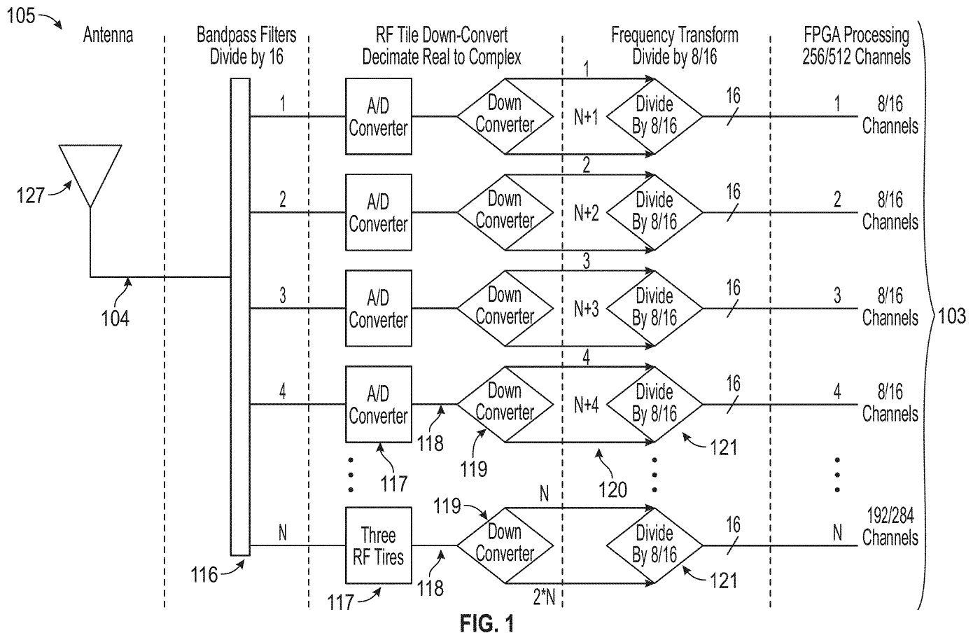

For example, in Examples 1 and 3 as shown below, it is demonstrated that that the “combination approach” is capable of detecting 1.26-6.62 fold copies of the fetal Y-Chr DNA marker (see FIG. 1 and Table 2), and is capable of detecting 1.34-1.98 fold copies of the HBV DNA marker (see FIG. 1 and Table 2), compared with the corresponding “traditional approach”. In another example, in Example 4 below, it is independently demonstrated that that the “combination approach” is capable of detecting 4.4-21.2 fold copies of the mRASSF 1 A marker (see Table 3), and is capable of detecting at least 14.5 fold copies of the mTERT marker (see Table 3), compared with the corresponding “traditional approach”.

In any embodiments of the method provided in this second aspect, the wash solution can have a pH of approximately 2.0-4.0, or can comprise a salt (NaCl, KCl, MgCl2, LiCl, sodium citrate, or any combination thereof) having a concentration of approximately 0.15-3 M.

In the following, a total of four specific examples are provided, which serve to further illustrate the inventions as disclosed herein, but by no means shall be construed as limiting the scope of in any way.

### Example 1

Briefly in this example, the impact of centrifugation-dependent preprocessing on the cfDNA recovery rate is evaluated, using the male fetal DNA detectable in the maternal urine or HBV DNA from patients with chronic HBV infection as indicators for transrenal DNA.

1. Materials and Methods

1.1 Subject and Urine Samples

For Y-Chr study, archived urine samples collected from various dates from two pregnant donors in the third trimester carrying a male fetus were used in this study.

For HBV-DNA study, archived urine samples collected from three chronic HBV infected donors each with HBV serum viral load at least 10⁶ IU/mL serum and detectable HBV DNA in urine measured from previous studies.

1.2 Urine Sample Collection

Briefly, a 50-ml urine sample was collected in EDTA-containing urine storage tubes at two different time points one-day apart. The urine sample collection is detailed in the following. Specifically, fresh collected urine was immediately mixed with 0.5 mol/L EDTA, pH 8.0, to a final concentration of 10-50 mmol/L EDTA to inhibit the possible nuclease activity in urine sample, and stored at −70° C. To isolate total urine DNA, frozen urine sample was thawed at room temperature, and then placed immediately in ice before DNA isolation. Thawed urine would be processed for DNA isolation within an hour.

1.3 Urine Sample Pre-Processing

15 ml of the above collected urine samples were first spiked with synthetic double stranded Spike-in (SPKN) DNA fragment at 10⁵ copies/mL and then pre-processed by centrifugation before DNA isolation. Briefly, for the pre-processing implicating centrifugation, the urine sample was centrifugated at 1,500 RPM at 4° C. for 10 minutes; the supernatant was collected to thereby be separated from the cell debris “pellet”. Each of two urine fractions (supernatant and pellet) underwent DNA isolation.

1.4 DNA Isolation

DNA was isolated from each of the above urine fractions, according to the total DNA isolation approach detailed in the following. Specifically, each urine sample was digested by proteinase K (1 mg/mL) in 2 M Guanidine Hydrochloride lysis buffer for 1 hour. The urine lysate was then mixed with 0.8 volume of 6 mol/L guanidine thiocyanate (Sigma, St. Louis, Mo.),1 volume of isopropanol and 50 uL of MagsilRed (Catalog #: A1641, Promega, Madison, Wis.) silica magnetic beads and incubated for overnight at room temperature with gentle rotating mixing. Beads-DNA complex was centrifuged, washed twice with 80% ethanol and DNA was eluted in 20 μl of water.

1.5 Real-Time PCR Quantification

Real-time PCR quantification of Y-Chromosome (“Chr”) DNA using Y-Chr qPCR assay (Lin et al. Diagnostics 2021. 11(4); Lin et al. Hepatology communication 2021) and HBV DNA using HBV pol/s assay, as detailed in Table 1. Sequences from the Y Chromosome (Y-Chr) or HBV DNA was amplified with the Roche LightCycler480 instrument platform by real-time quantitative PCR to quantify the amounts of isolated fetal DNA or HBV DNA in the different urine fractions. The Y-Chromosome DNA quantification kit (detailed in Table 1) and HBV pol/s DNA quantification kit (detailed in Table 1) were used according to the protocol where each fraction was quantified in duplicates in a 15 μl containing 6 μl of urine DNA sample. The synthetic Spike-in SPKN DNA was quantified using the JBS Artificial Spike-In (SPKN) DNA Quantification kit (detailed in Table 1) according to the manufacturer's specifications, and the recovered copies were estimated in each cfDNA sample.

TABLE 1
Primers, probes sequences and PCR condition for the marker assays

| Target | Primer | Sequence | Target Sequence | PCR condition/ |
|---|---|---|---|---|
| Gene | oligos | (5‘-3‘) | GRCh38/hg38 | Amplicon Size |
| Y-Chr | Forward | CATCCAGAGCGTCCCTGGCTT | ChrY: 6246721- | 95° C.-10 sec, |
| | Primer | (SEQ ID NO: 1) | 6246780 | 61° C.-10 sec; 72° C. |
| | Reverse | GGCCGAAGAAACACTGAGAA | | -10 sec, 45 cycles/ |
| | Primer | (SEQ ID NO: 2) | | 60 bp |
| Pol/s(CG1) | Forward | TTGGGACACCGGAAAGATGTAAG | NC_003977.1 | 95° C. 5 mins (95° C. |
| | Primer | ATGAGTCTAGACTCGTGGTG | | 10 sec, 60° C. |
| | | (SEQ ID NO: 3) | | 10 sec, 72° C. |
| | Reverse | AGAACATCCGGATCTATATGGTCT | nt 246-286 | 10 sec) × 45 cycles/ |
| | Primer | GCCCCTAGAAAATTGAGAGAAGTC | | 41 bp |
| | | (SEQ ID NO: 4) | | |
| SPKN | Forward | GGACGGAGATGAAGGTCTACTA | GACCGTCAATTCACCGAGGA | 95° C. 5 min (95° C.- |
| | Primer | (SEQ ID NO: 5) | CGGAGATGAAGGTCTACT | 10 sec, 60° C.- |
| | Reverse | GGGAGGTCTCCACCCTATTTA | ACTCTCCGTACTGCCGAGGG | 10 sec, 72° C.- |
| | Primer | (SEQ ID NO: 6) | GCCAGATAACGGTTGAATTC | 10 sec) × 45 cycles/ |
| | | | GTGAGAGCTATCCCCTAAAT | 97 bp |
| | | | AGGGTGGAGACCTCCCTC | |
| | | | GGTGAATTGACGGTC (SEQ | |
| | | | ID NO: 7, where the primer | |
| | | | recognition region is | |
| | | | underlined and in bold) | |
| hTERT 124 | Forward | AGGGGCTGGGAGGGC(SEQ ID | GRCh38/hg38 | 95° C. 5 min, (95° C. |
| (m TERT) | Primer | NO: 8) | Chr5:1295093- | 10 sec, 80° C. 5 s, |
| | (TERT_F) | | 1295138 | 68° C.* 5 sec, 60° C. |
| | Reverse | CCTCCCGGGTCCCCG (SEQ ID | | 1 sec) × 50 cycles/ |
| | Primer | NO: 9) | | 46 bp |
| | (TERT_R) | | | |
| | BNA clamp | G+CC+C+C+C+T+CC+GG-PH | | |
| | | (SEQ ID NO: 10*) | | |
| | Probe | FAM-CA+GC+CCC+TTC+CG | | |
| | | (SEQ ID NO: 11**) | | |
| mRASSF1A | Forward | AAATACGGGTATTTTCGC (SEQ ID | GRCh38/hg38 | 95° C.-5 min (95° C. |
| | Primer | NO: 12) | Chr3:50341063- | -10 sec, 56° C.- |
| | Reverse | AACGACCACAACGACG(SEQ ID | 50341114 | 10 sec, 72° C.*- |
| | Primer | NO: 13) | | 10 sec) × 45 cycles/ |
| | Probe 1 | FAM-T+GT+TTTG+CGG+TCG+T | | 52 bp |
| | | (SEQ ID NO: 14**) | | |
| K-RAS | 1^{st}-step | GCTCTTCGTGGTGTGGTGTCCATA | GRCh38/hg38 | 95° C. 15 min (hotstart |
| | Forward | TAAACTTGTGGTAGTTGGACCT | Chr12: 25245294- | Taq), (94° C. 30 sec, |
| | Primer | (SEQ ID NO: 15) | 25245381 | 52° C. 30 sec) × 20 |
| | 1^{st}-step | GCTCTTCGTGGTGTGGTGTCCCG | | cycle/ 129 bp |
| | Reverse | TCCACAAAATGATTCTGA (SEQ ID | | 95° C. for 15 min |
| | Primer | NO: 16) | | (94° C. |
| | 2^{nd}-step | ACTGAATATAAACTTGTGGTAGTT | | 30 sec, 56° C. 30 sec)/ |
| | Forward | GGACCT (SEQ ID NO: 17) | | 87 bp |
| | Primer | | | |
| | 2^{nd}-step | GTCCACAAAATGATCCTGGAT | | |
| | Reverse | TAGC (SEQ ID NO: 18) | | |
| | Primer | | | |

*The nucleotides with italicized fonts and having “+” immediately on their left denote bridged nucleic acid (BNA) bases; and “PH” denotes a phosphorylation modification.
**The nucleotides with italicized fonts and having “+” immediately on their left denote locked nucleic acid (LNA) bases; and “FAM” denotes a fluorescein.

The SPKN recovery from input was calculated using the formula (1):

Total Output/Total Input×100%=% SPKN from Input; (1)

The Y-Chr or HBV DNA distribution in supernatant or pellet fraction was calculated using the formula (2):

“Pellet” or “Supernatant”/(Pellet+Supernatant)×100%; (2)

In the study with Y-Chr DNA shown in FIG. 1, an exogenous SPKN 131 bp dsDNA (SEQ ID NO: 7) was used as a control for the fractionation of supernatant and cell pellet by centrifugation. As expected, in the top panel of FIG. 1, SPKN was recovered mostly from the supernatant fraction ranging from 48%-99%, while in contrast, the recovery of SPKN DNA from the pellet fraction was found to be quite minimal, ranging from 1%-7.2%. Interestingly and unexpectedly, as shown in the bottom panel of FIG. 1, while the amounts of the Y-Chr DNA that can be recovered from the pellet fraction varies among different samples, they are commonly much more significant than the SPKN DNA control, which notably rangs from 21%-88% of total detected Y-Chr.

This result indicates that unlike the exogenously added SPKN DNA, there is an unexpected association of Y-Chr DNA with the cell debris pellet. This further suggests that the centrifugation preprocessing, which is commonly performed in almost all existing cfDNA isolation and detection methods, will lose a significant proportion of cfDNAs from the circulation or transrenal DNA due to their association to the cell debris fraction in the post-centrifugation pellet fraction. Thus if a method, such as the method described in the second aspect of the disclosure as described above that recovers the cfDNA from the post-centrifugation “Pellet” fraction in addition to the regular post-centrifugation “Supernant” fraction, there will be substantially significantly more cfDNA recovered, leading to the detection of significantly more copies of the Y-Chr DNA marker. If the “Pellet” fraction is combined with the “Supernant” fraction, there will be estimately 1.26 fold (i.e. 1/79.4%), 5.24 fold (i.e. 1/19.1%), 1.54 fold (i.e. 1/64.9%), 6.62 fold (i.e. 1/15.1%), 2.50 fold (i.e. 1/39.9%) and 1.72 fold (i.e. 1/58.2%), respectively for the six test groups shown in FIG. 1, of the Y-Chr DNA marker compared with the “Supernatant” fraction only.

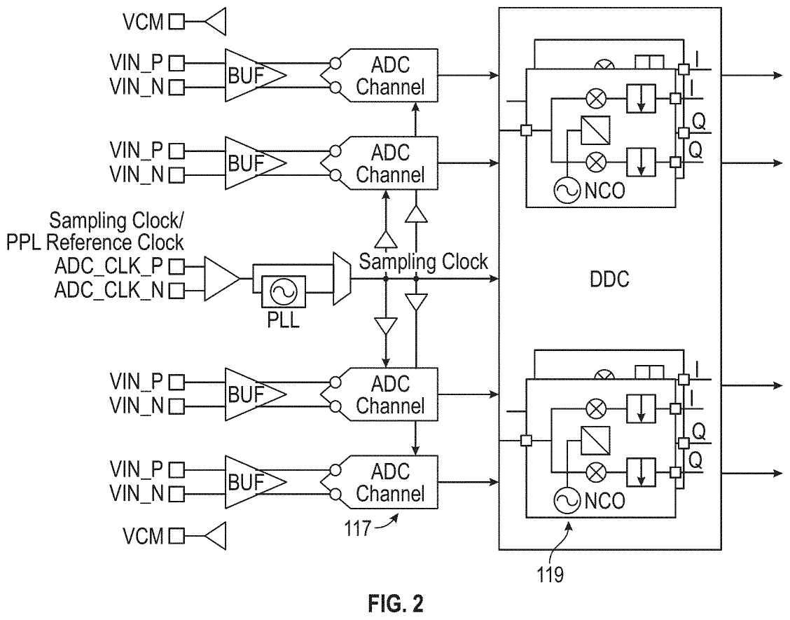

In the study with HBV DNA shown in FIG. 2, the above mentioned exogenous spiked 131 bp dsDNA (SEQ ID NO: 7) was also used as a control for the fractionation of supernatant and cell pellet by centrifugation. As expected, shown in the top panel of FIG. 2, SPKN was recovered mostly from the supernatant fraction ranging from 77%-82%, and only low amount (ranging from 1.5%-21.4%) of SPKN DNA was recovered from the pellet fraction. Interestingly, unexpectedly, yet notably also in line with the above Y-Chr DNA results, a significant amount of HBV DNA was recovered from the cell debris pellets ranging from 26%-50% of total detected HBV DNA, as shown in the bottom panel of FIG. 2, once again indicating that that a significant amount of HBV DNA can be associated with cell debris pellet which could have been lost in almost all existing cfDNA isolation and detection methods which concern only the supernatant fraction after the centrifugation preprocessing step.

Thus, similar to the fetal Y-Chr DNA marker detection results shown in FIG. 1, the combination of the “Pellet” fraction with the traditional “Supernatant” fraction is expected to be able to detect approximately 1.84 fold (i.e. 1/54.3%), 1.34 fold (i.e. 1/74.5%) and 1.98 fold (i.e. 1/50.4%) respectively for the three test groups shown in FIG. 2, of the HBV marker compared with the “Supernatant” fraction only.

### Example 2

Removal of High-MW DNA by gel eletrophoresis Enhanced the Detection of Mutated K-ras DNA in Urine of Patients with Colorectal Diseases

To detect K-ras codon 12 mutations, restriction endonuclease-enriched polymerase chain reaction (RE-PCR) was performed as previously described (Su et al. 2004) on total and low-MW urine DNA (specific primers and assay conditions shown in Table 1).

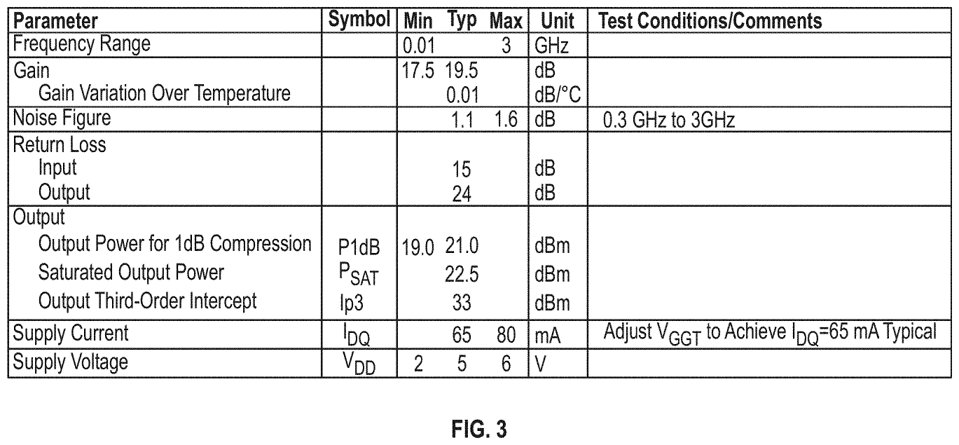

In brief, total DNA or fractionated low-MW DNA, derived from 200 μL urine was used in each assay. The PCR product from the RE-PCR assay is 87 bp, and the appearance of a 71-bp fragment after the second BstNI digestion is evidence of mutated K-ras DNA in the DNA sample. The detection limit of the RE-PCR assay is 15 copies of the mutant K-ras per 100 ng of wild-type DNA per reaction (Su et al. 2004). As assay controls, DNA prepared from sources known to have either mutant (human adeno-carcinoma SW480 cells) or wild-type (human hepatoblastoma HepG2 cells) K-ras sequences was subjected to PCR. As expected, and shown in FIG. 3, DNA prepared from HepG2 cells contained no detectable levels of mutant K-ras (no appearance of the 71-bp fragment after the second BstNI digestion), whereas DNA prepared from SW480 cells contained mutant K-ras sequences (the 71-bp fragment was detected after the second BstNI digestion). For each urine sample that contained detectable mutant K-ras DNA, the presence of the mutant K-ras sequence was more evident when low-MW DNA was used in the assay as compared to the total urine DNA, as demonstrated by the six different individuals (i.e. “FX”, “GD”, “GG”, “GI”, “GM”, and “GN”) in FIG. 3.

In order to estimate the enhancement of detection sensitivity by LMW DNA fractionation, an end-point detection was performed by a 2-fold serial dilution. Briefly, both total DNA and LMW DNA was first subjected to five 2-fold serial dilutions, (1:2, 1:4, 1:8, 1:16, and 1:32). Each dilution was subjected RE-PCR assay to determine the end-point of dilution that contained detectable K-ras mutation. The fold increase of Low/Total DNA calculated and indicated in FIG. 3. The detection sensitivity increases in a range of 4-16 fold. This is quite unexpected because the processing step of LMW DNA fractionation usually causes the loss of cfDNA from total DNA. As noted, the K-ras RE-PCR assay is not a robust assay and its analytic sensitivity decreased approximately 10-20 fold with additional of 100 ng human wild-type background DNA (Su et al. 2004), thus removal of HMW DNA enhanced the detection sensitivity. The fold increase of detecting mutated K-ras DNA in low-MW (LMW) DNA per total urine DNA were calculated and indicated in FIG. 3. There are ranging from 4-16 folds increase of detection sensitivity by using Low-MW urine DNA as substrate for K-ras mutation detection as compared to using total urine DNA. This indicates the significant improvement for K-ras mutation detection in urine if high molecular weight (>1 kb) was removed by LMW DNA fractionation.

### Example 3

Briefly in this example, the impact of centrifugation-dependent preprocessing on the cfDNA recovery rate is evaluated, using the male fetal DNA detectable in the maternal urine as an indicator to suggest that the associated Y-Chr DNA can be wash-off from the cell pellet.

1. Materials and Methods

1.1 Subject and Urine Samples

Archived urine sample collected from a single pregnant donor in the third trimester carrying a male fetus was used in this study.

1.2 Urine Sample Collection

Briefly, a 50-ml urine sample was collected in EDTA-containing urine storage tubes at two different time points one-day apart. The urine sample collection is detailed in the following. Specifically, fresh collected urine was immediately mixed with 0.5 mol/L EDTA, pH 8.0, to a final concentration of 10-50 mmol/L EDTA to inhibit the possible nuclease activity in urine sample, and stored at −70° C. To isolate total urine DNA, frozen urine sample was thawed at room temperature, and then placed immediately in ice before DNA isolation. Thawed urine would be processed for DNA isolation within an hour.

1.3 Urine Sample Pre-Processing

15 ml of the above collected urine sample was pre-processed by either centrifugation or no centrifugation (short as “N.C.” hereinafter) before DNA isolation. Briefly, for the pre-processing implicating centrifugation, the urine sample was centrifugated at 1,500 RPM at 4° C. for 10 minutes; the supernatant was collected to thereby be separated from the cell debris pellet, which is termed “pre-wash pellet” to differentiate from “post-wash pellet” below, and the supernatant was then put on ice; the pre-wash pellet was then washed briefly with 1 ml of sodium citrate buffer (pH 3.0); the wash-off solution was collected by centrifugation at 13,000 RPM for 90 s; then each of the three urine fractions (supernatant, wash-off, and post-wash pellet) urine fraction underwent DNA isolation. For the pre-processing implicating no centrifugation (i.e. the N.C. approach), the urine sample directly underwent DNA isolation and Low-MW DNA fractionation by using carboxylated beads.

1.4 DNA Isolation

DNA was isolated from each of the above urine fractions or from the N.C. urine sample according to the total DNA isolation approach detailed in the following. Specifically, each urine sample was digested by proteinase K (1 mg/mL) in 2 M Guanidine Hydrochloride lysis buffer for 1 hour. The urine lysate was then mixed with 0.8 volume of 6 mol/L guanidine thiocyanate (Sigma, St. Louis, Mo.),1 volume of isopropanol and 50 uL of MagsilRed (Catalog #: A1641, Promega, Madison, Wis.) silica magnetic beads and incubated for overnight at room temperature with gentle rotating mixing. Beads-DNA complex was centrifuged, washed twice with 80% ethanol and DNA was eluted in 20 μl of water.

1.5 Real-Time PCR Quantification of Y-Chromosome (“Chr”) DNA Using Y-Chr qPCR Assay

Sequences from the Y Chromosome (Y-Chr) was amplified with the Roche LightCycler480 instrument platform by real-time quantitative PCR, as detailed in Table 1, to quantify the amounts of isolated fetal DNA in the different urine fractions. The Y-Chromosome DNA quantification kit, as detailed in Table 1 (JBS Science, Doylestown, Pa.) was used according to the protocol where each fraction was quantified in duplicates in a 15 μl containing 6 pit of urine DNA sample.

2. Results

Table 2 summarizes the detection results (i.e. results for the capabilities in detecting the circulating cell-free fetal DNA from the maternal urine samples, using the copies of circulating cell-free fetal DNA marker “Y-Chr” as indicators) for each fraction/sample, specifically including the “Supernatant”, “Wash-off”, and “Post-wash Pellet” fractions for the centrifugation pre-processed urine samples and the no-centrifugation pre-processed urine samples (i.e. the N.C. urine samples), at each of the two different urine collection timepoints (i.e. Collections #1 and 2).

As shown, the “Supernatant” fraction contains approximately 7.99 and 1.51 (for the Collections #1 and #2 respectively) copies of detectable Y-Chr DNAs, which substantially represent the amounts of target DNA in the urine sample that can be detectable by the traditional centrifugation-relying approach which usually concerns only the post-centrifugation supernatant fraction.

Surprisingly and unexpectedly, when the usually discarded pellet fraction after centrifugation was washed, the resulting “Wash-off” fraction contains approximately 2.78 and 1.91 (for the Collections #1 and #2 respectively) copies of Y-Chr DNAs, and the “Post-wash Pellet” fraction contains no detectable such target DNAs. When the copies of Y-Chr DNAs from the “Supernatant” fraction and from the “Wash-off” fraction are combined to thereby obtain the total copies of the Y-Chr DNAs that are recovered (i.e. “Total recovery after centrifugation”, 10.77 and 3.42 copies for the Collections #1 and #2 respectively), such recovered Y-Chr DNA is approximately 1.4 and 2.3 (for the Collections #1 and #2 respectively) fold compared with the “Supernatant” only. Thus it can be seen that compared with the traditional DNA isolation method which concerns only the post-centrifugation supernatant fraction, there is surprisingly a significant proportion of the target Y-Chr DNAs left in the usually discarded post-centrifugation pellet fraction, which would have been otherwise lost and is estimated to be approximately 25.8%-55.8% of the total target DNA combining the DNAs obtained from the post-centrifugation supernatant fraction and the DNAs from the post-centrifugation pellet fraction (i.e. for Collection #1: 2.78/10.77=25.8%; and for Collection #2: 1.91/3.42=55.8%).

Even more surprisingly and unexpectedly, if the DNAs were directly isolated from the urine samples with the usual centrifugation preprocessing step being totally skipped or removed, the copies of target DNAs that can be detected thereby are dramatically much more than the target Y-Chr DNAs detected from the post-centrifugation supernatant fraction. To be more specific, there are 49.58 and 17.62 (for the Collections #1 and #2 respectively) copies of Y-Chr DNA that are detectable in the “No-Centrifugation” or “N.C.” group, which are thus notably 6.2 and 11.7 (for the Collections #1 and #2 respectively) fold compared with the “Supernatant” only group. Thus it can be seen that as much as 83.9%-91.4% of the target Y-Chr DNA could have been lost if the traditional centrifugation-implicated way of DNA isolation is performed which concerns only the post-centrifugation supernatant fraction, compared with the no-centrifugation approach disclosed herein (i.e. for Collection #1: (49.58-7.99)/49.58=83.9%; and for Collection #2: (17.62-1.51)/17.62=91.4%). In other words, compared with the traditional cfDNA isolation method that requires the removal of the cell debris from the urine sample (e.g. by centrifugation preprocessing), the method disclosed herein, which substantially removes the cell debris-removal preprocessing, can isolate approximately 5.2-10.7 fold more of detectable target Y-Chr DNA, thus dramatically increasing the detection sensitivity and dramatically lowering the detection limit for such target low-molecular weight (LMW) DNAs in the urine sample.

TABLE 2
Fetal Y-Chromosome detection (copies/mL urine) in various isolated pregnant (PG) urine
DNA fractions.

| | Centrifugation | | | | No-Centrifugation |
|---|---|---|---|---|---|
| | | | | Total Recovery | (N.C.) |
| | | | | (fold change | (fold change |
| | | | | relative to | relative to |
| | | | Post-wash | “Supernatant” | “Supernatant” |
| | Supernatant | Wash-off | Pellet | only) | only) |
| Collection #1 | 7.99 | 2.78 | ND | 10.77 (1.4 fold) | 49.58 (6.2 fold) |
| Collection #2 | 1.51 | 1.91 | ND | 3.42 (2.3 fold) | 17.62 (11.7 fold) |

“ND”, not detectable.

### Example 4

Association of HCC-associated DNA markers with cell debris pellet.

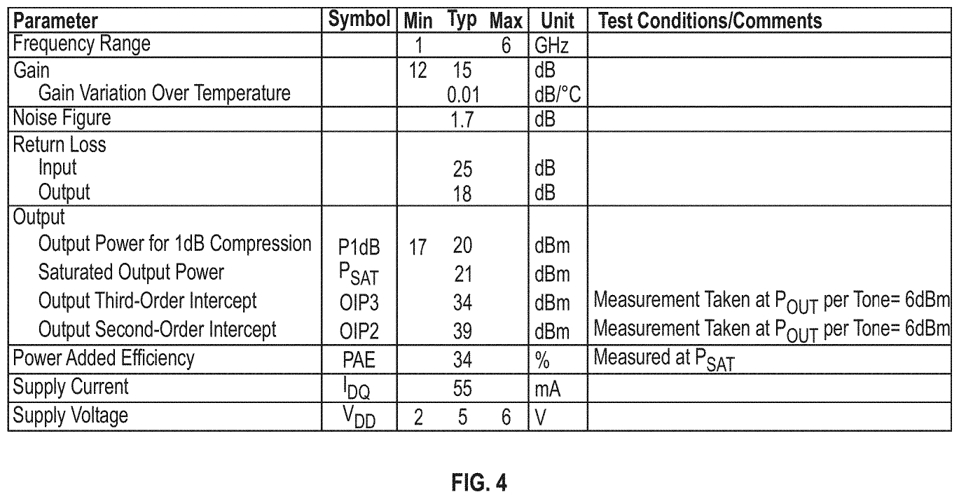

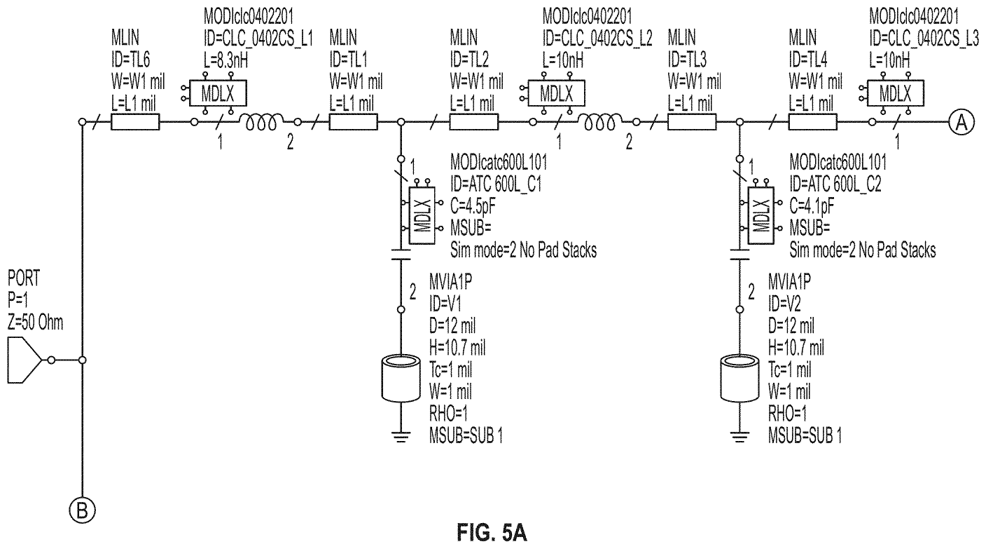

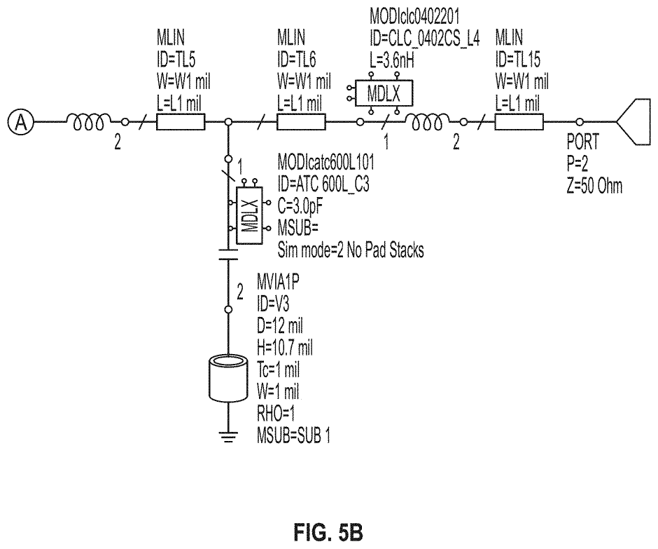

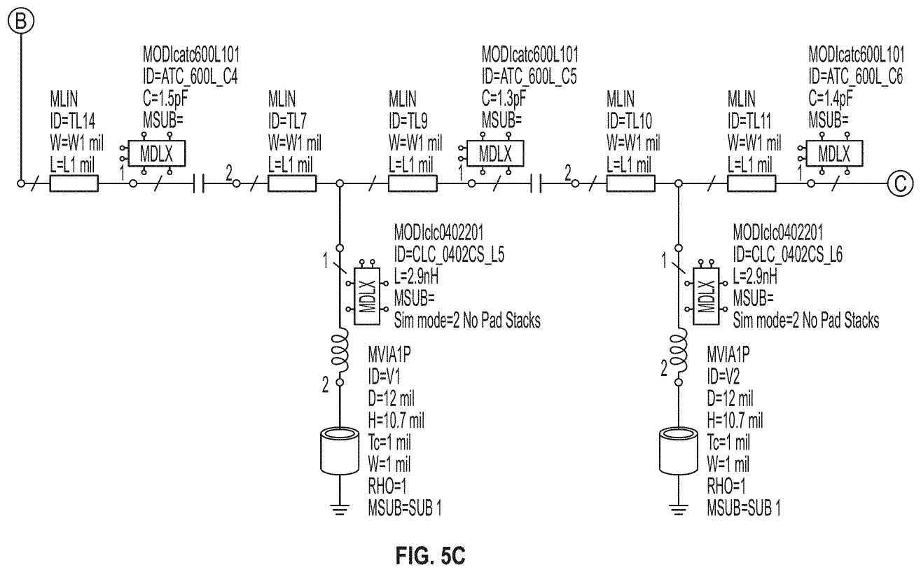

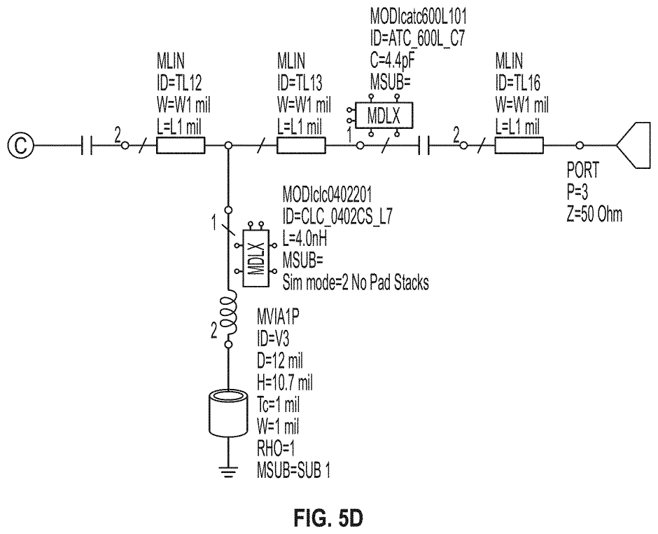

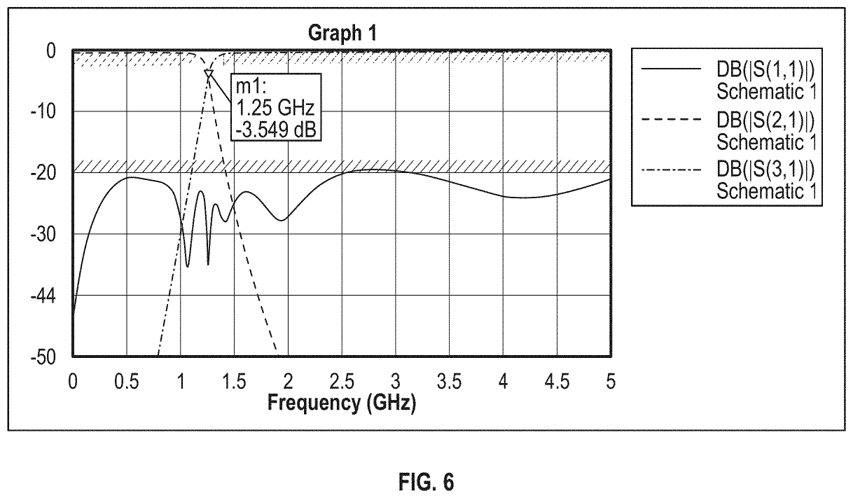

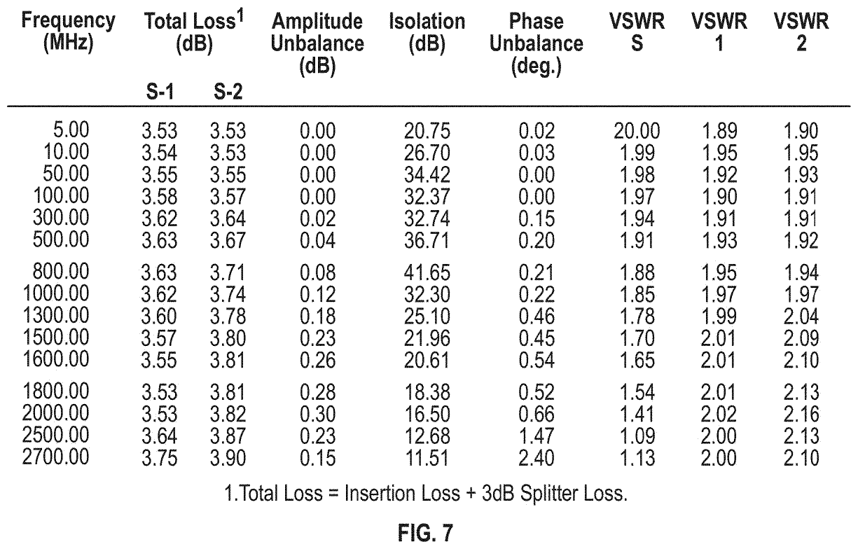

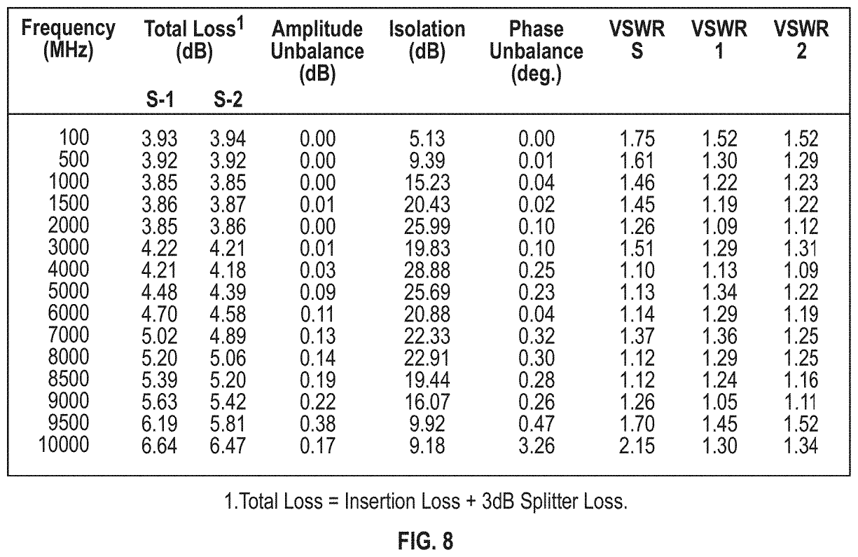

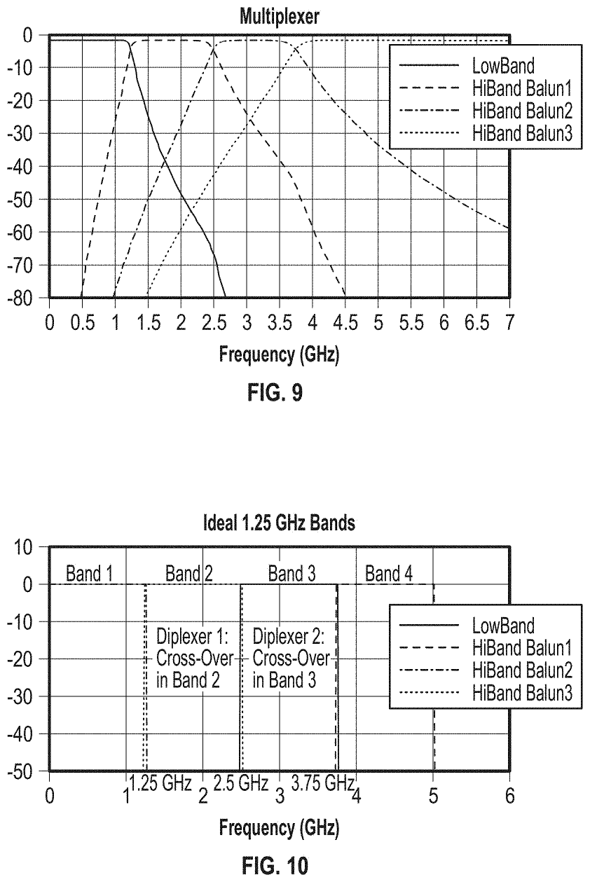

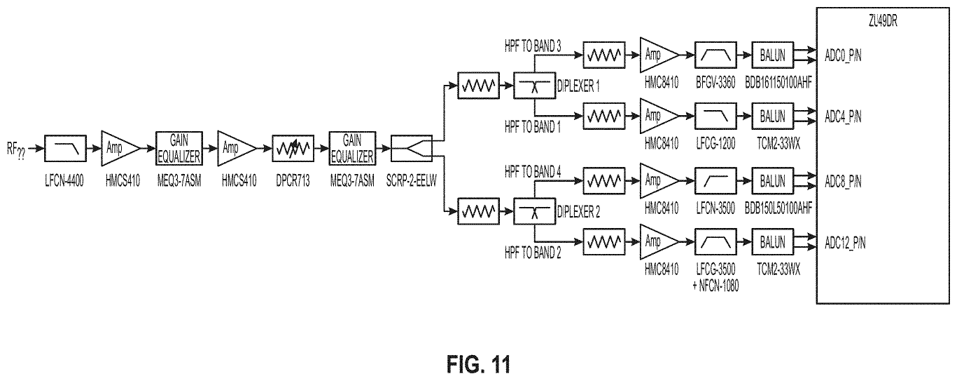

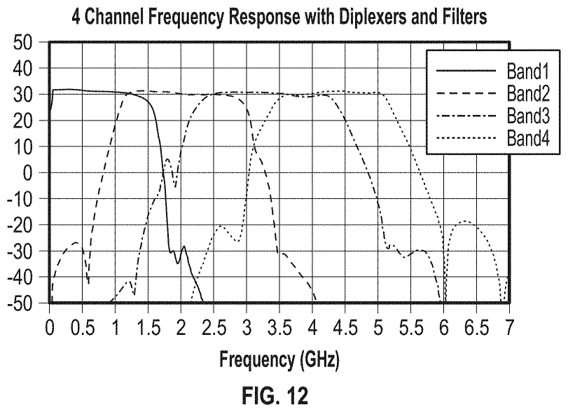

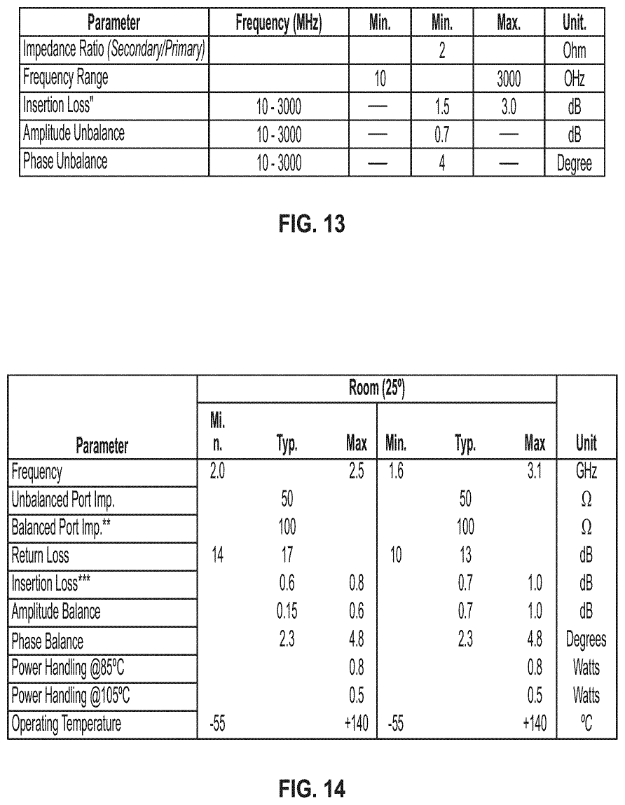

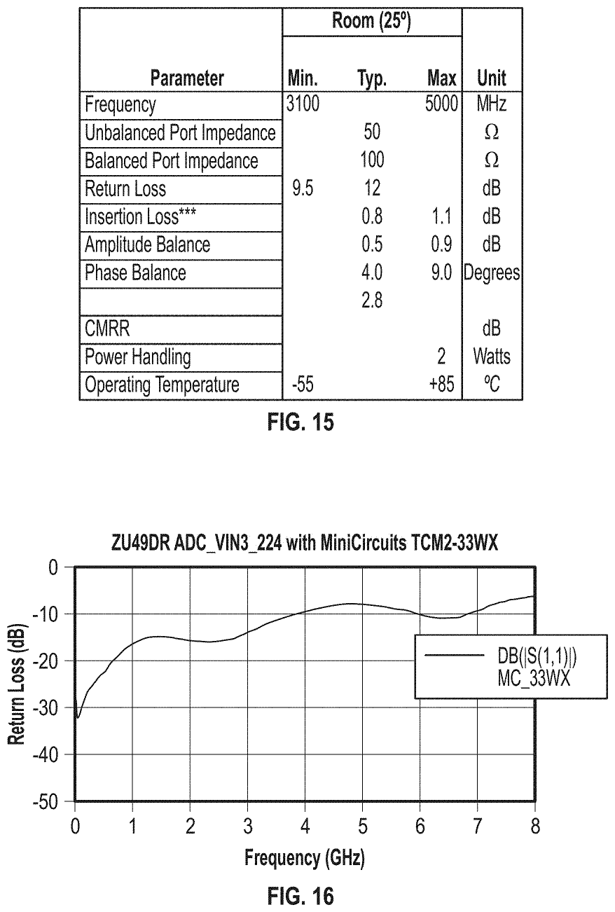

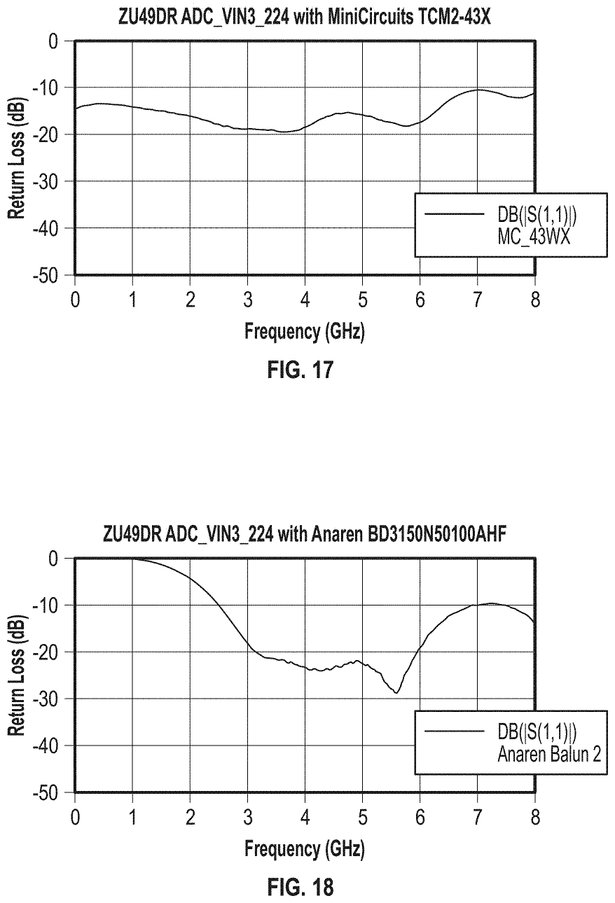

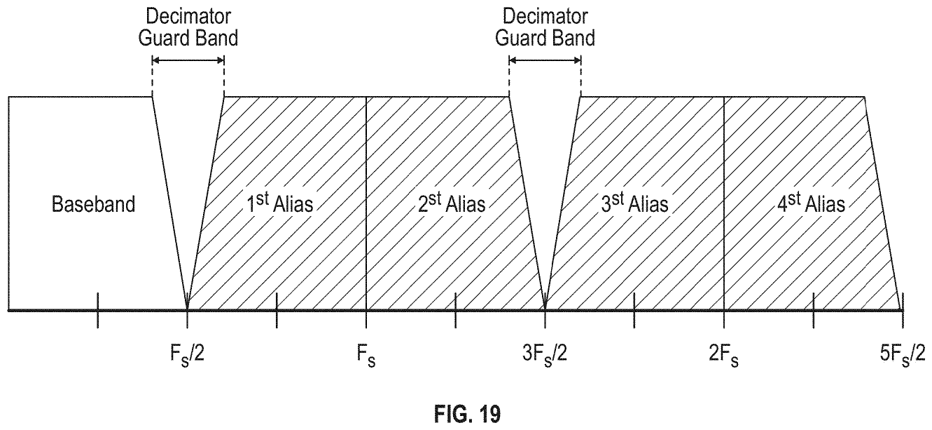

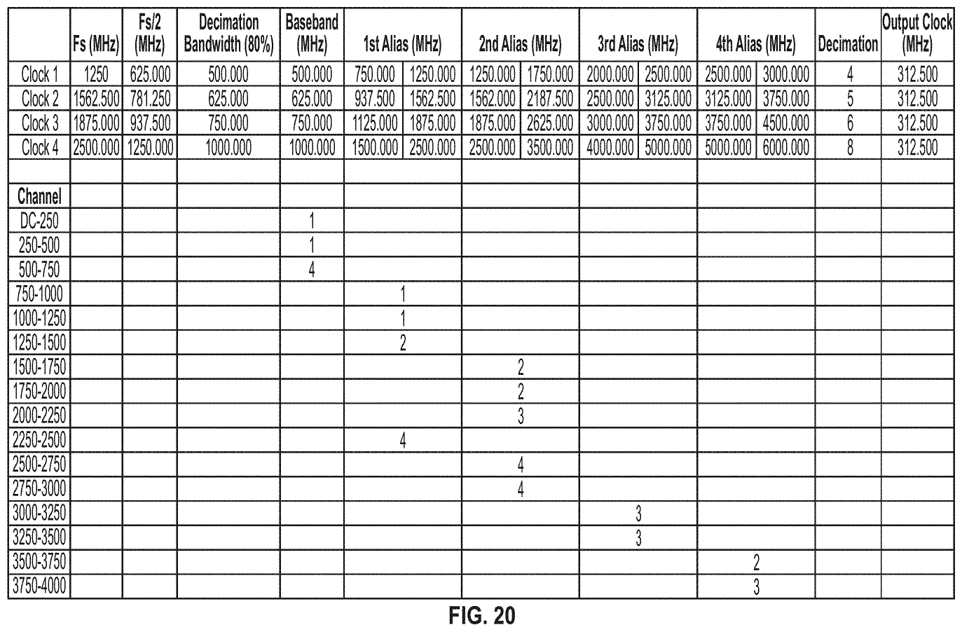

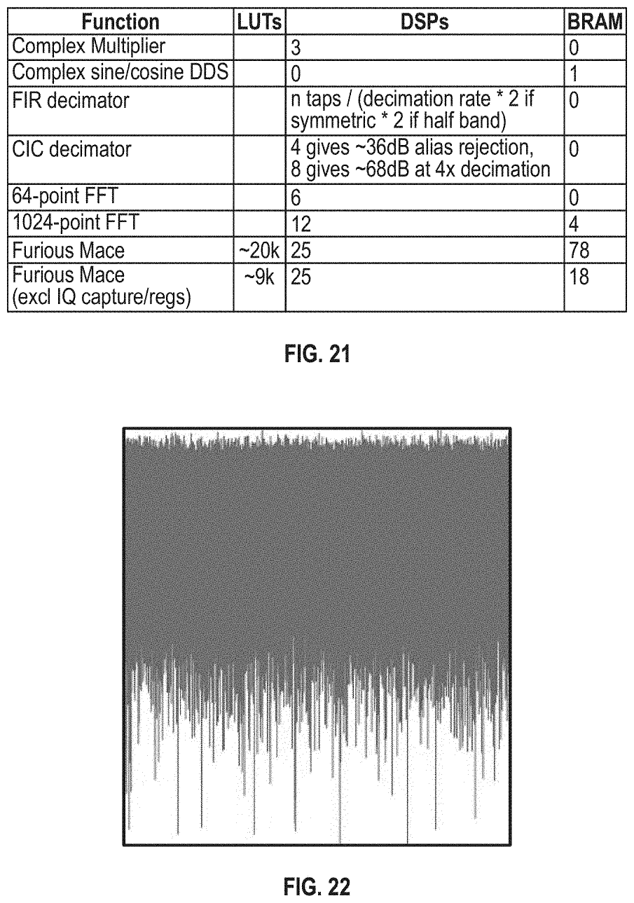

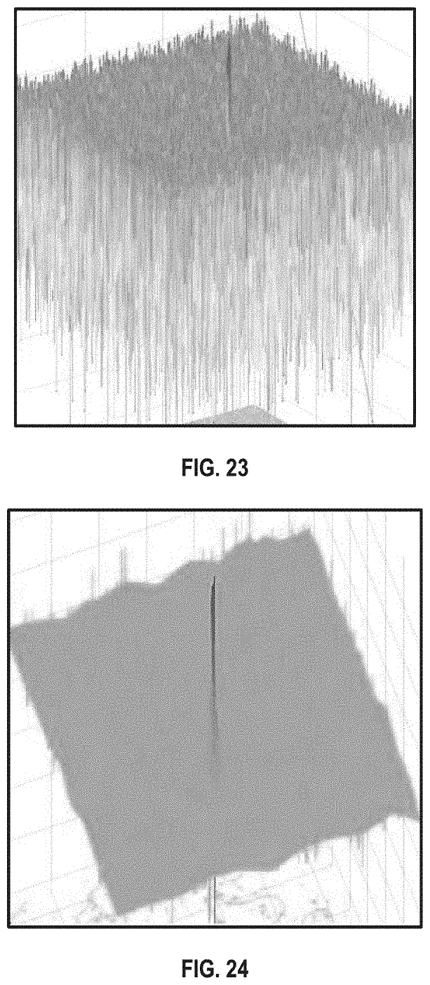

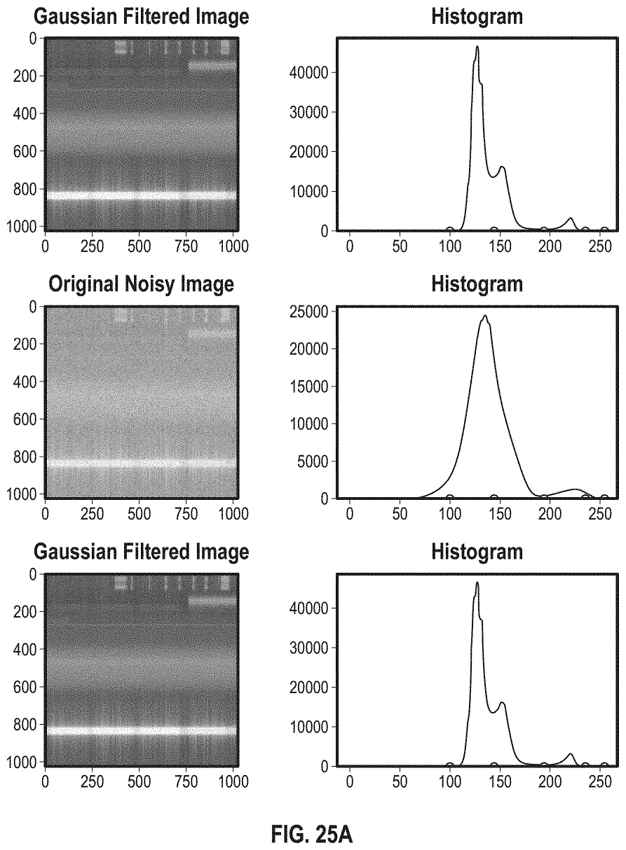

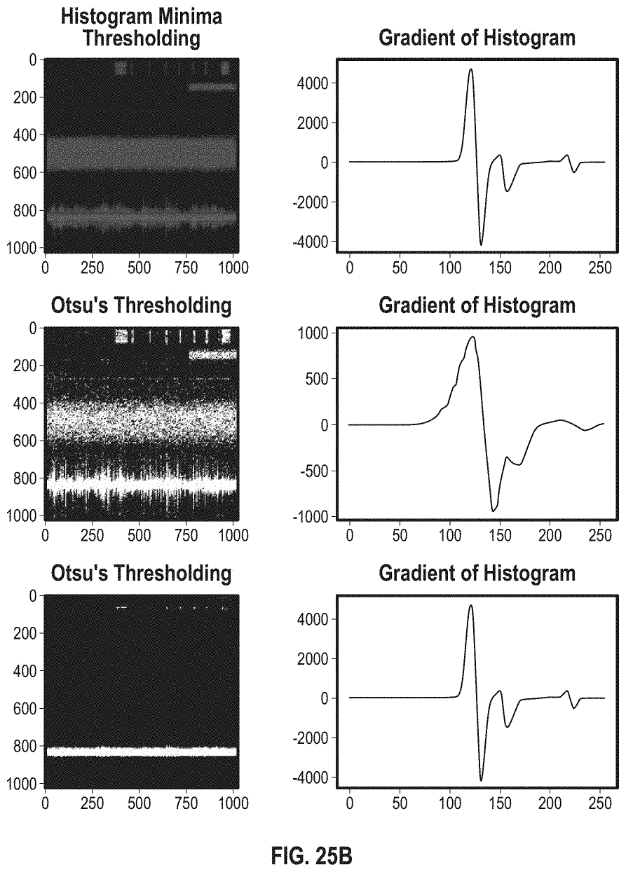

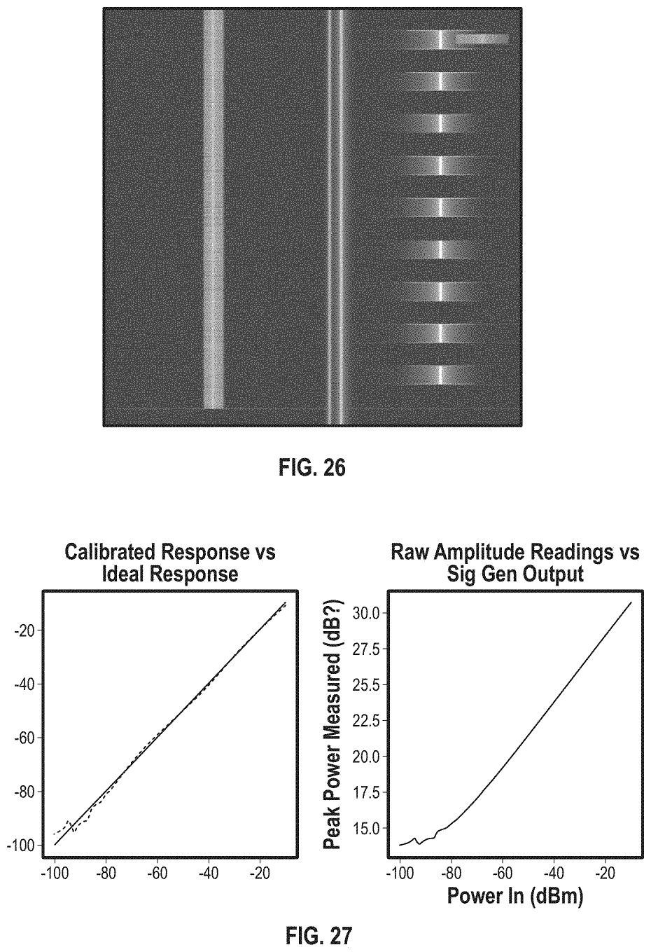

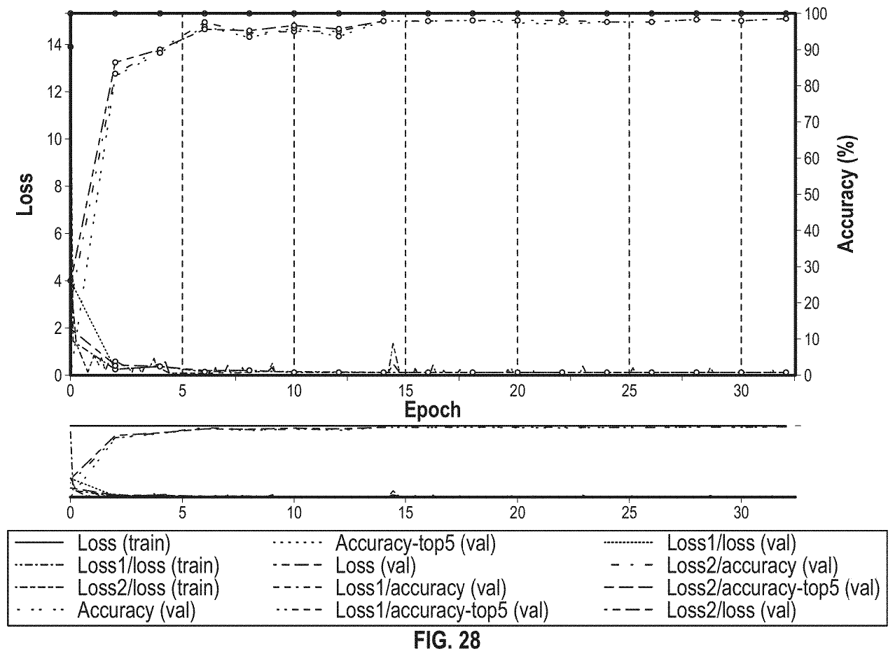

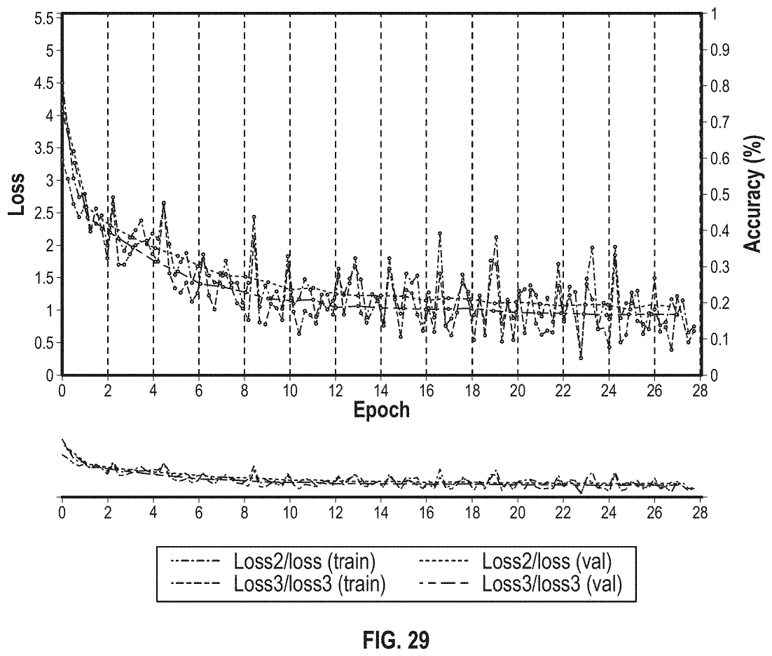

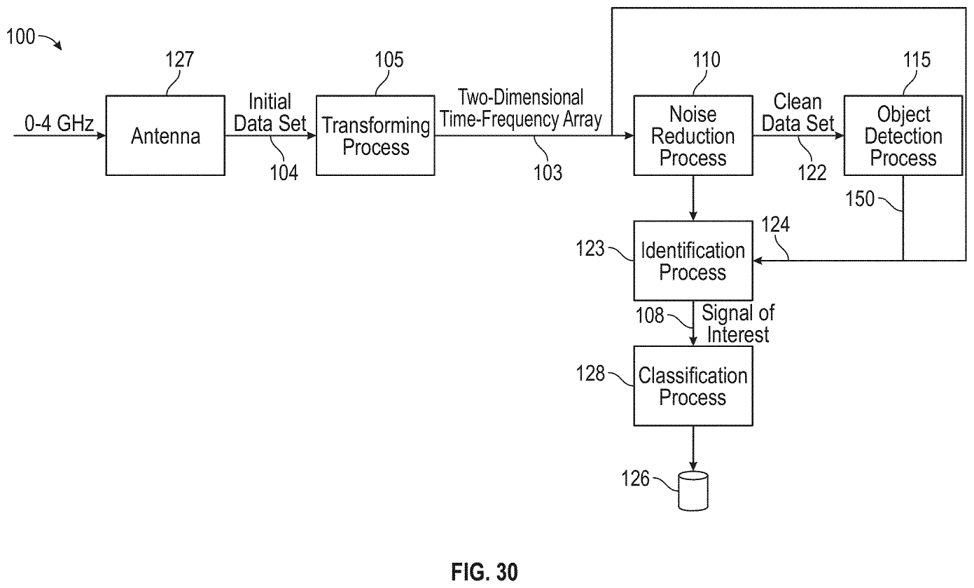

Urine collected from patients with two known detectable HCC DNA markers in urine. Different urine aliquots, 10 mL each, were subjected to total urine DNA isolation without centrifugation and then fraction to obtain low molecular weight (LMW) DNA that is shorter than 1 kb (i.e. “No-Centrifugation” or N.C. group), or centrifugation pre-processing and DNA isolation as described above to obtain DNA from either supernatant (Sup) or pellet fraction (i.e. “Centrifugation” group), as illustrated in FIG. 4. DNA was then subjected to methylated RASSF1A (mRASSF1A) and hTERT-124 mutation (i.e. mTERT) marker assays as detailed in Table 1. The summary results are shown in Table 3.

TABLE 3
HCC marker (mRASSF1A and mTERT) detection in urine samples by different methods.

| | | Centrifugation | | | No-Centrifugation | |
|---|---|---|---|---|---|---|
| | | | | Total Recovery | (N.C.) | |
| | | | | (fold change | (fold change | |
| | | | | relative to | relative to | |
| | Patient | | | “Supernatant” | “Supernatant” | |
| | # | Supernatant | Pellet | only**) | only**) | |
| mRASSF1A | #1 | 1.6 | 32.3 | 33.9 (21.2 fold) | 46.7 | (29.2 fold) |
| (copies/mL) | #2 | 1.6 | 5.4 | 7.0 (4.4 fold) | 11.4 | (7.1 fold) |
| mTERT | #3 | <1 (BLOD) | <1 (BLOD) | NA* | 6 | (>6 fold) |
| (copies/10 ng) | #4 | <1 (BLOD) | 13.5 | ~14.5 (>14.5 fold) | 16.5 | (>16.5 fold) |

BLOD: Below limit of detection, defined as less than 3 copies per 3,000 TERT DNA input (10 ng)
*not available
**the fold change is calculated assuming the max value (i.e. 1) for the “supernatant” fraction

As shown in Table 3, the mRASSF1A marker was reproducibly detected in both the “Centrifugation” and “No-Centrifugation” group of urine aliquots although the quantities of the marker varied. When the traditional cfDNA isolation method was applied (i.e. corresponding the “Supernatant” fraction in the “Centrifugation” group), there was only 1.6 copies of the mRASSF1A marker detected, and similar to the observation described above for the Y-Chr shown in Table 2, there were a lot more such marker detected in the “Pellet” fraction in the “Centrifugation” group (32.3 and 5.4 copies for the Patient #1 and #2 respectively). Thus if the “Supernatant” fraction and the “Pellet” fraction are combined, the total detectable level of the mRASSF1A marker (i.e. “Total Recovery”) would be approximately 21.2 and 4.4 (for the Patient #1 and #2 respectively) fold compared with the traditional method that concerns only the post-centrifugation “Supernatant” fraction. Furthermore, even more dramatically, if the assay was performed without centrifugation (i.e. the “No-Centrifugation” or “N.C.” group), the total detectable level of the mRASSF1A marker was approximately 29.2 and 7.1 (for the Patient #1 and #2 respectively) fold compared with the traditional method that concerns only the post-centrifugation “Supernatant” fraction. Such results align very well with the fetal Y-Chr DNA detection result shown in above Table 2.

Regarding the mTERT marker, it is shown to be below limit of detection (i.e. BLOD) in the post-centrifugation “Supernanat” fraction in both of the two patients (#3 and #4), indicating that the mTERT marker is substantially non-detectable by means of the traditional centrifugation-implicated cfDNA isolation and detection assay. In patient #3, although it is also shown to be below limit of detection (i.e. BLOD) in the post-centrifugation “Pellet” fraction, if the mTERT marker is detected by an assay without centrifugation (i.e. the “No-Centrifugation” or “N.C.” group), there were approximately 6 copies of the mTERT marker detected, which is at least 6 fold compared with the “Supernatant” group. In patient #4, there were significantly more copies (i.e. 13.5) of the marker detected in the post-centrifugation “Pellet” fraction, and even more copies (i.e. 16.5) were detected in the “N.C.” group. Thus the data on both patients further indicated that if the assay was performed without centrifugation, the total detectable level of the mTERT marker was more than 6 fold compared with the traditional method that substantially concerns only the post-centrifugation “Supernatant” fraction.

HCC-associated DNA markers were detected in every pellet fraction. This data indicates that the pre-processing centrifugation of removing cell pellet from urine cfDNA isolation can lose trans-renal DNA of interest. Since different aliquots of urine from same individual can contain different amount of cell debris, DNA marker quantities between aliquots from the same patient was not compared.

### DISCUSSION

As shown in Examples 1 and 2, transrenal fetal cfDNA is detectable in almost all samples or fractions of the maternal urine samples except for the post-wash cell pellet sample from Example 2, Table 1. Even more surprisingly, shown in the example 2, the “Wash-off” fraction contains a significant amount of fetal cfDNA, which is at least ⅓ of the “Supernatant” fraction in the Collection #1 sample, and is even 1.26 fold of the “Supernatant” fraction in the Collection #2 sample. These results demonstrate that the pre-wash pellet (in Example 2) or pellet (in Example 1) fraction, which is substantially the typically discarded pellet after the traditional centrifugation pre-processing of a urine sample, contains a surprisingly significant portion of the fetal cfDNA. This indicates that some of transrenal circulatory extracellular DNA molecules associate with the cells or cell debris in the collected urine sample, which are pelleted down with the cell pellet after centrifugation at the typical pre-processing centrifugation speed of 1,000 rpm for 10 min or a higher speed for shorter time.

As further indicated from Table 2, the N.C. approach surprisingly seems to achieve a much higher efficiency in recovering the fetal cfDNA, an unambiguous transrenal DNA marker, from the maternal urine sample than the centrifugation-wash-off approach, because the Y-Chr copies recovered in the N.C. sample is around 4-5-fold compared with the Y-Chr copies in the centrifugation pre-processed sample in all the fractions combined. For example, for collection #1, the difference between N.C. sample (i.e. 49.58) and the combined centrifugation preprocessed sample (i.e. 7.99+2.78=10.77) is around 4.6 fold. For collection #2, the difference between N.C. sample (i.e. 17.62) and the combined centrifugation preprocessed sample (i.e. 1.51+1.91=3.42) is around 5.2 fold. It is speculated that it is due to the loss of DNA in each pre-processing including wash-off of the pellet and isolation step for the latter. As such, the direct DNA isolation from the collected urine sample without pre-processing centrifugation represent a preferred approach for recovering transrenal cfDNA from urine samples. Of note, exogenous spiked cfDNA such as dsDNA SPKN 131 bp DNA fragment has little or no significant association with cell pellet and remained in the supernatant which can be used as fractionation control for supernatant from cell pellet. This indicates that not all species of cfDNA associates to the same extent with cell debris pellet generated by pre-processing centrifugation. Example 3 provides another transrenal cfDNA, HCC-associated DNA markers which were also detected in pellet fractions after pre-processing centrifugation. Centrifugation as a pre-processing step can similarly cause a significant portion of HCC-derived DNA markers or other intrinsic cell-free nucleic acids (cfNAs), such as miRNAs and cfRNAs etc., in addition to the cfDNAs to be pulled down with the pellet obtained thereby, given their similar physiochemical properties as cfDNAs.

### CONCLUSION

An examination of circulatory extracellular fetal DNA and liver derived DNA including DNA from hepatocellular carcinoma and HBV DNA in urine, has shown surprisingly two important observations. First, a significant amount of circulation derived cell-free DNA (cfDNA) in urine or transrenal DNA is associated with cell pellet generated by centrifugation. As a result, pre-processing using centrifugation to obtain supernatant for urine cfDNA isolation can lose a significant amount of cfDNAs of interest such as fetal DNA, HCC DNA, colorectal cancer DNA or HBV DNA or other transrenal DNA of interest, if DNA isolation is performed only from the supernatant. Second, the circulation derived cfDNA in urine or transrenal DNA has a relatively small size of less than 1 kb, low-molecular weight (LMW) DNA, whereas the genomic DNA obtained inside of cell is much larger, i.e. high-molecular weight (HMW) DNA. To isolate cfDNA in body fluid, such as in urine, without re-processing, by isolating total urine DNA followed by removal of HMW DNA, bulk of background DNA, the DNA of interest can be increase of concentration, thus enhance of detection sensitivity. This selective enrichment of circulation derived cfDNA in urine or other body fluid by size fraction to remove HMW DNA to obtain LMW DNA can be accomplished by variety of methods, including but not limited to: using chromatography or electrophoresis such as chromatography on agarose or polyacrylamide gels, ion-pair reversed-phase high performance liquid chromatography, capillary electrophoresis in a self-coating, low-viscosity or other polymer matrix, selective extraction in microfabricated electrophoresis devices, microchip electrophoresis, adsorptive membrane chromatography, and the like; density gradient centrifiguation, and methods utilizing nanotechnological means sucha s microfabricated entropic trap arrays, carboxylated beads, and the like.

The LMW DNA fractionation thus obtained without centrifugation or other means to collect cell debris, by isolating total DNA and then removal of HMW DNA, not only permits the subsequent determination of fetal genetic traits in pregnancies at risk for detecting genetic disorder, sex, or cancer genetics for early cancer detection, cancer screening, and disease management, or viral genetics for disease management.

Such determination of genetics of interest can be effected by methods such as, for example, polymerase chain reaction (PCR) technology, probe hybridizations, next generation sequencing, nucleic acid arrays (such as DNA chips) and the like.

### REFERENCES

1. Chan, A. K., R. W. Chiu, and Y. D. Lo, Cell-free nucleic acids in plasma, serum and urine: a new tool in molecular diagnosis. Annals of clinical biochemistry, 2003. 40(2): p. 122-130.
2. Tsui, N. B., et al., High resolution size analysis of fetal DNA in the urine of pregnant women by paired-end massively parallel sequencing. PloS one, 2012. 7(10): p. e48319.
3. Botezatu, I., et al., Genetic analysis of DNA excreted in urine: a new approach for detecting specific genomic DNA sequences from cells dying in an organism. Clinical chemistry, 2000. 46(8): p. 1078-1084.
4. Al-Yatama, M. K., et al., Detection of Y chromosome-specific DNA in the plasma and urine of pregnant women using nested polymerase chain reaction. Prenatal diagnosis, 2001. 21(5): p. 399-402.
5. Majer, S., et al., Maternal urine for prenatal diagnosis—an analysis of cell-free fetal DNA in maternal urine and plasma in the third trimester. Prenatal diagnosis, 2007. 27(13): p. 1219-1223.
6. Li, Y., et al., Inability to detect cell free fetal DNA in the urine of normal pregnant women nor in those affected by preeclampsia associated HELLP syndrome. Journal of the society for gynecologic investigation, 2003. 10(8): p. 503-508.
7. Illanes, S., et al., Detection of cell-free fetal DNA in maternal urine. Prenatal Diagnosis, 2006. 26(13): p. 1216-1218.
8. Koide, K., et al., Fragmentation of cell-free fetal DNA in plasma and urine of pregnant women. Prenatal diagnosis, 2005. 25(7): p. 604-607.
9. Shekhtman, E. M., et al., Optimization of transrenal DNA analysis: detection of fetal DNA in maternal urine. Clinical chemistry, 2009. 55(4): p. 723-729.
10. Lin, S. Y., et al., A New Method for Improving Extraction Efficiency and Purity of Urine and Plasma Cell-Free DNA. Diagnostics, 2021. 11(4): p. 650-659.
11. Lin, S. Y., et al., Detection of Hepatitis B Virus—Host Junction Sequences in Urine of Infected Patients. Hepatology communication, 2021. 5(10): p. 1649-1659.
12. Jain, S., et al., Characterization of the hepatitis B virus DNA detected in urine of chronic hepatitis B patients. BMC gastroenterology, 2018. 18(1): p. 40.
13. Lin, S. Y., et al., Detection of CTNNB1 Hotspot Mutations in Cell-Free DNA from the Urine of Hepatocellular Carcinoma Patients. Diagnostics, 2021. 11(8): p. 1475.
14. Hann, H. W., et al., Detection of urine DNA markers for monitoring recurrent hepatocellular carcinoma. Hepatoma research, 2017. 3: p. 105-111.
15. Zhang, A., et al. Urine as an Alternative to Blood for Cancer Liquid Biopsy and Precision Medicine. in 2018 IEEE International Conference on Bioinformatics and Biomedicine (BIBM). 2018. IEEE.
16. Kim, A. K., et al., Urine DNA biomarkers for hepatocellular carcinoma screening. British journal of cancer, 2022. 126: p. 1432-1438.
17. Su, Y. H., et al., Human urine contains small, 150 to 250 nucleotide-sized, soluble DNA derived from the circulation and may be useful in the detection of colorectal cancer. Journal of nolecular diagnostics, 2004. 6(2): p. 101-107.
18. Su, Y. H., et al., Detection of K-ras mutation in urine of patients with colorectal cancer. Cancer biomarkers, 2005. 1: p. 177-182.
19. Song, B. P., et al., Detection of hypermethylated vimentin in urine of patients with colorectal cancer. Journal of molecular diagnostics, 2012. 14(2): p. 112-119.

1. A method for characterizing a target cell-free nucleic acid molecule present in a biological sample, the method comprising:
isolating total cell-free nucleic acids from the biological sample without prior preprocessing the biological sample that removes cell debris therefrom; and
characterizing the target cell-free nucleic acid molecule from the isolated total cell-free nucleic acids;
wherein:
the method is capable of detecting at least 2 fold copies of the target cell-free nuclei acid molecule in the biological sample compared with when the cell debris is removed in the prior preprocessing in the isolating step.

2. The method of claim 1, wherein the biological sample comprises a urine sample, a serum sample, a plasma sample, a saliva sample, a sweat sample, a lymph liquid sample, or any combination thereof.

3. The method of claim 2, wherein the biological sample is a urine sample, and the target cell-free nucleic acid molecule comprises at least one of a transrenal nuclei acid molecule or an apoptosis-derived nucleic acid molecule.

4. The method of claim 1, wherein the target cell-free nucleic acid molecule is a target cell-free DNA molecule, wherein:
the isolating total cell-free nucleic acids from the biological sample comprises:
isolating total cell-free DNAs from the urine sample; and
the characterizing the target cell-free nucleic acid molecule from the isolated total cell-free nucleic acids comprises:
characterizing the target cell-free DNA molecule from the isolated total cell-free DNAs.

5. The method of claim 4, wherein the target cell-free DNA molecule has a length shorter than approximately 1 kb, wherein:
the method further comprises, after the isolating total cell-free DNAs from the urine sample and before the characterizing the target cell-free DNA molecule from the isolated total cell-free DNAs:
obtaining low-molecular weight DNAs from the isolated total cell-free DNAs;
wherein:
the characterizing the target cell-free DNA molecule from the isolated total cell-free DNAs comprises:
characterizing the target cell-free DNA molecule from the low-molecular weight DNAs.

6. The method of claim 5, wherein the obtaining low-molecular weight DNAs from the isolated total DNAs is by means of a size differentiation approach, selected from a carboxylated magnetic beads-based approach, a chromatography approach based on an agarose gel, or a chromatography approach based on a polyacrylamide gel.

7. The method of claim 5, wherein the characterizing the target cell-free DNA molecule from the low-molecular weight DNAs is by means of at least one of a polymerase chain reaction (PCR) assay, a sequencing assay, or a hybridization assay.

8. The method of claim 5, wherein the target cell-free DNA molecule is a cancer-associated DNA marker, selected from a mutant K-ras, methylated RASSF1A (mRASSF1A), or mutated TERT (mTERT).

9. The method of claim 5, wherein the urine sample is obtained from a pregnant female, and the target cell-free DNA molecule is a fetal DNA marker that is associated with sex, an autosomal trait, or a genetic disorder.

10. The method of claim 9, wherein the fetal DNA marker comprises a Y-Chromosome (Y-Chr) marker.

11. The method of claim 5, wherein the target cell-free DNA molecule is a DNA marker for a micro-organism, wherein the micro-organism is a virus, a bacterium, or a fungus.

12. The method of claim 11, wherein the target cell-free DNA molecule is a DNA marker for hepatitis B virus (HBV).

13. The method of claim 1, wherein the target cell-free nucleic acid molecule is a target cell-free RNA molecule, wherein:
the isolating total cell-free nucleic acids from the biological sample comprises:
isolating total cell-free RNAs from the urine sample;
and
the characterizing the target cell-free nucleic acid molecule from the isolated total cell-free nucleic acids comprises:
characterizing the target cell-free RNA molecule from the isolated total cell-free RNAs.

14. The method of claim 13, wherein the target cell-free RNA molecule comprises a microRNA.

15. The method of claim 1, wherein the isolating total cell-free nucleic acids from the biological sample is performed with use of carrier RNAs.

16. The method of claim 1, wherein the method is capable of detecting at least 6 fold copies of the target cell-free nuclei acid molecule in the biological sample compared with when the cell debris is removed in the prior preprocessing in the isolating step.

17. A method for characterizing a target cell-free nucleic acid molecule in a biological sample, the method comprising:
preprocessing the biological sample, comprising:
centrifugating the biological sample to thereby obtain a supernatant fraction and a pellet fraction;
washing the pellet fraction using a wash solution to thereby obtain a wash-off fraction; and
combining the supernatant fraction and the wash-off fraction to thereby obtain a combined fraction;
isolating total cell-free nucleic acids from the combined fraction; and
characterizing the target cell-free nucleic acid molecule from the isolated total cell-free nucleic acids;
wherein:
the method is capable of detecting at least 1.25 fold copies of the target cell-free nucleic acid molecule from the biological sample compared with when the total cell-free nucleic acids are isolated only from the supernatant fraction in the preprocessed biological sample.

18. The method of claim 17, wherein the wash solution has a pH of approximately 2.0-4.0.

19. The method of claim 17, wherein the wash solution comprises a salt having a concentration of approximately 0.15-3 M, wherein the salt is NaCl, KCl, MgCl₂, LiCl, sodium citrate, or any combination thereof.

20. The method of claim 17, wherein the method is capable of detecting at least 2 fold copies of the target cell-free nucleic acid molecule from the biological sample compared with when the total cell-free nucleic acids are isolated only from the supernatant fraction in the preprocessed biological sample.
